SEQUENCE LISTING (4 sequences)

SEQ ID NO: 1 - protein, 5 aa, synthetic construct
  SITE 5: note = 5-Amino-4-oxo-1,2,4,5,6,7-hexahydroazepino[3,2,1-hi ]indole-2-carboxylic acid
EGGIX

SEQ ID NO: 2 - protein, 5 aa, synthetic construct
  SITE 5: note = 5-Amino-4-oxo-1,2,4,5,6,7-hexahydroazepino[3,2,1-hi ]indole-2-carboxylic acid
EGGIX

SEQ ID NO: 3 - protein, 5 aa, synthetic construct
  SITE 5: note = 5-Amino-4-oxo-1,2,4,5,6,7-hexahydroazepino[3,2,1-hi ]indole-2-carboxylic acid
EGGIX

SEQ ID NO: 4 - protein, 5 aa, synthetic construct
  SITE 5: note = 5-Amino-4-oxo-1,2,4,5,6,7-hexahydroazepino[3,2,1-hi ]indole-2-carboxylic acid
EGGIX

(19) United States
(12) Patent Application Publication
(10) Pub. No.: US 2025/0064991 A1
(43) Pub. Date: Feb. 27, 2025

(54) COMPOUNDS SPECIFIC TO GRANZYME B AND USES THEREOF

(71) Applicant: CytoSite BioPharma Inc., Sudbury, MA (US)

(72) Inventors: Geoffrey Malcolm BILCER, Shorewood, MN (US); Hui XIONG, Indianapolis, IN (US); Carey HORCHLER, Indianapolis, IN (US); Mark A. CASTANARES, Indianapolis, IN (US); Brian LIEBERMAN, Indianapolis, IN (US); Juntian ZHANG, Indianapolis, IN (US); Francisco A. VALENZUELA, Indianapolis, IN (US)

(73) Assignee: CytoSite Biopharma Inc., Sudbury, MA (US)

(21) Appl. No.: 18/736,979

(22) Filed: Jun. 7, 2024

Related U.S. Application Data
(60) Provisional application No. 63/506,718, filed on Jun. 7, 2023.

(51) Int. Cl. A61K 51/04; A61K 45/06; C07B 59/00

(52) U.S. Cl. CPC A61K 51/0468; A61K 45/06; C07B 59/002; A61K 2121/00; A61K 2123/00; C07B 2200/05

(57) ABSTRACT

Compounds capable of binding to granzyme B and comprising a radioactive moiety, for examples, compounds of Formula (I), and pharmaceutical compositions comprising such. Also provided herein are uses of the compounds and pharmaceutical composition in cancer treatment and/or imaging.

### CROSS-REFERENCE TO RELATED APPLICATIONS

This application claims the benefit of U.S. Provisional Application No. 63/506,718, filed Jun. 7, 2023, the content of which is incorporated by reference herein in its entirety.

### SEQUENCE LISTING

The instant application contains a Sequence Listing which has been submitted electronically in XML file format and is hereby incorporated by reference in its entirety. Said XML copy, created on Oct. 21, 2024, is named 101720-0066_8009_USOO_SL.xml and is 6,206 bytes in size.

### TECHNICAL FIELD

This disclosure relates to radioactive compounds that are useful as a therapeutic agent, and more particularly to radioactive compounds that are useful for identifying Granzyme B and eliminate cancer cells containing such.

### BACKGROUND

Granzyme B is a serine-protease most commonly found in the granules of natural killer cells and cytotoxic T cells. Granzyme B is released along with the pore-forming protein perforin at the immunological-synapse formed between T-cells and their targets. A portion of the released Granzyme B then enters cancer cells, primarily through perforin-pores, where it activates multiple substrates leading to activation of the caspase cascade. As a downstream effector of tumoral cytotoxic T cells, granzyme B has been used as an early biomarker for tumors responding to immunotherapy.

There is a need to develop new compounds that act as effective Granzyme B imaging agents and therapies for treating immunoregulatory abnormality such as cancer.

### SUMMARY

The present application provides radioactive compounds capable of targeting Granzyme B and uses thereof as a therapeutic agent for treating diseases associated with Granzyme B such as cancer.

In some aspects, provided herein is a compound having a structure of Formula (I) or a stereoisomer, tautomer, or a salt thereof.

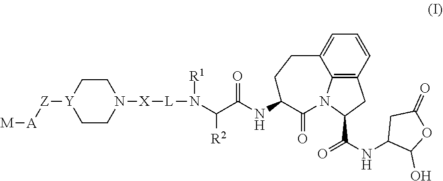

In Formula (I),

M represents a radioactive moiety;

A is a chelating moiety chelating the radioactive moiety of M;

X is —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;

Y is CH or N;

Z is —CH₂—, —CH₂C(NH)—, —CH₂C(O)—, —CH₂C(S)—, —NHC(NH)—, —NHC(O)—, —NHC(S)—, —OC(NH)—, —OC(O)—, or —OC(S)—; optionally wherein Z is —CH₂— or —CH²C(O)—;

L is a peptide linker having 1-6 amino acid residues, inclusive;

R¹ is H or C₁₋₆ alkyl, optionally wherein R¹ is H or methyl; and

R² is C₁₋₆ alkyl or C₃₋₆ cycloalkyl.

In some embodiments, the compound is of Formula (Ia):

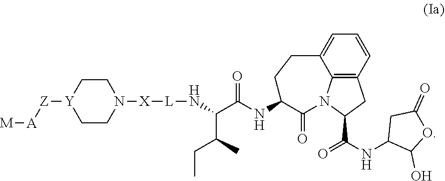

In any of the compounds of Formula (I), e.g., compounds of Formula (Ia), X can be —CH₂C(O)—.

In some instances, the compound is of Formula (Ib):

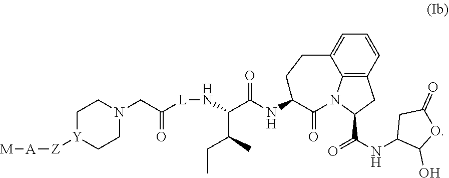

In any of the compounds of Formula (I), e.g., compounds or Formula (Ia) or Formula (Ib) provided herein, L may be a peptide having 1-5 amino acid residues (1, 2, 3, 4, or 5, inclusive). In some instances, L may have one or more non-naturally occurring amino acid residues. Exemplary peptides of L are provided below: Glu-Gly-Gly, Glu-βAla-βAla, γGlu, _{D}∵Glu, γGlu-βAla, _{D}Glu-βAla-βAla, _{D}Glu-AEA, _{D}Glu-AEEA-AEEA, _{D}Glu-_{D}Glu-AEA, _{D}Glu-DGlu-βAla-βAla, βAla-_{D}Glu-βAla, Diacid-βAla-βAla, N-acid-βAla-βAla, or βAla-N-acid-βAla.

In any of the compounds of Formula (I), e.g., compounds or Formula (Ia) or Formula (Ib) provided herein, the chelating moiety A may be 1,4,7-triazacyclononane-N,N′,N″-triacetic acid (NOTA), 1,4,7-triazacyclononane-4,7-diyl diacetic acid (NODA), 1,4,7,10-tetraazacyclododecane-1,4,7,10-tetraacetic acid (DOTA), 1,4,7,10-Tetraazacyclododecane-1,4,7-triacetic acid (DO3A), Restrained Complexing Agent (RESCA), or MACROPA. In any of the compounds of Formula (I), e.g., compounds or Formula (Ia) or Formula (Ib) provided herein, the radioactive moiety of M may be a therapeutic radioisotope. Examples include, but are not limited to, ⁶⁷Cu, ⁹⁰Y, ¹⁷⁷Lu, ²²⁵Ac, ⁴⁷Sc, ¹³¹I, ¹⁵³Sm, ¹⁶¹Tb, ²¹¹At, ²¹²Pb, ²¹²Bi, ²²³Ra, or ^{227Th}. In some specific examples, the therapeutic radioisotope is ⁹⁰Y.

In some embodiments, the chelating moiety is NOTA or DOTA in the compound provided herein and the therapeutic radioisotope is ⁹⁰Y, ¹⁷⁷Lu, or ²²⁵Ac. In other examples, the chelating moiety is NODA, and the therapeutic radioisotope is ⁴⁷Sc or ⁶⁷Cu.

In some examples, the compound disclosed herein has the following structure:

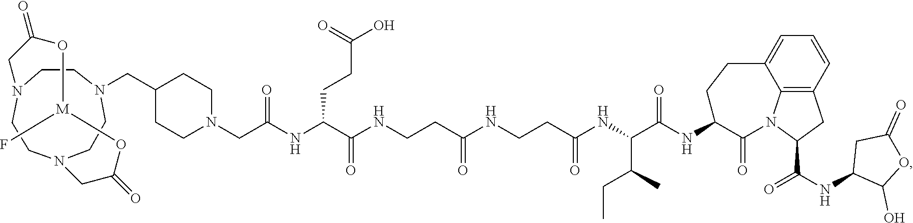

in which M is ¹⁷⁷Lu, ⁹⁰Y, ²²⁵Ac, or ²¹³Bi.

In other examples, the compound disclosed herein has the following structure:

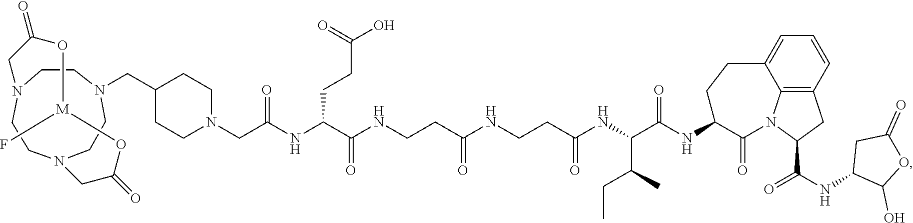

in which M is ¹⁷⁷Lu, ⁹⁰Y, ²²⁵Ac, or ²¹³Bi.

Pharmaceutical compositions comprising one or more of the foregoing compounds of Formula (I) are also provided and within the scope of the present disclosure.

In other aspects, the present disclosure features a method of treating cancer in a subject, the method comprising administering to a subject in need thereof an effective amount of any of the Formula (I) compounds disclosed herein or a pharmaceutical composition comprising such as also disclosed herein.

In some instances, the subject can be administered an immunotherapeutic agent prior to receiving the compound of Formula (I). Exemplary immunotherapeutic agents include, but are not limited to, an immune checkpoint inhibitor (e.g., a PD1 inhibitor such as an anti-PD1 or anti-PD-L1 antibody), or a genetically engineered T cell expressing a chimeric antigen receptor (CAR).

Alternatively or in addition, the subject can be administered an imaging agent to image granzyme B.

In some examples, any of the methods disclosed herein may further comprise treating the subject with one or more additional therapeutic agents, for example, anti-inflammatory agents, steroids, immunotherapy agents, and/or chemotherapeutic agents.

Also within the scope of the present disclosures are pharmaceutical compositions comprising any of the Formula (I) compounds (e.g., Formula (Ia) and Formula (Ib) compounds) disclosed herein for use in cancer therapy, as well as uses of any of the Formula (I) compounds for manufacturing a medicament for cancer treatment.

Various aspects and embodiments now will be described more fully hereinafter. Such aspects and embodiments may take many different forms, and the exemplary ones disclosed herein should not be construed as limiting; rather, these embodiments are provided so that this disclosure will be thorough and complete, and will fully convey its scope to those skilled in the art.

### DETAILED DESCRIPTION

Cancer immunotherapies have represented a significant advance in cancer therapy over recent years. Antibodies directed against immune checkpoints such as programmed cell death protein 1 (PD-1) and cytotoxic t lymphocyte-associated protein 4 (CTLA-4) have been approved with positive outcomes for some patients. Research into the field of immune-oncology continues, with strategies including CAR-T cells, vaccines, small molecules, and antibodies under development. Despite the promise of these therapies, they are not a panacea. These immunotherapies can be associated with significant adverse events, which are costly, and the response rates are typically 20-50%, meaning the majority of patients do not respond to therapy. Furthermore, determining an individual patient's response to therapy can be challenging using conventional methods, as response is frequently associated with an immune-cell infiltrate that can make responding tumors appear to grow on anatomic imaging (e.g., CT, MRI) and demonstrate increased avidity with FDG-PET imaging due to the influx of metabolically active immune cells. Given the constraints of current imaging technologies, clinical studies for cancer immunotherapies typically employ overall survival as their study endpoint as opposed to progression-free survival.

Theranostic refers to a combination of diagnostic imaging and therapy for diagnosing and treating a target disease. In general, agents used in theranostic therapy contain a diagnostic imaging compound and a radio-active therapeutic compound. In some instances, the diagnostic imaging and therapeutic compound may have the same molecule except for different radio-labels, one nuclide for imaging purposes and one for treatment purposes. In some instances, the diagnostic imaging and therapeutic compound may have different elements bound to the same pharmacophore or molecule with different radioisotopes for imaging purposes and for treatment purposes.

Granzyme B, a downstream marker of cytotoxic T-cell activity, could serve as a novel biomarker to assess cancer immunotherapy efficacy. Granzyme B expression within a tumor can be assessed not only for CTL presence or absence, but also as an effector protein released by active T-cells that also integrates a measure of CTL activity, thus accounting for issues of T-cell exhaustion that make assessment of CTL presence difficult to accomplish. The present disclosure provides certain specific compounds capable of binding to Granzyme B (GZB), e.g., Formula (I) compounds such as Formula (Ia) and Formula (Ib) compounds, which show high binding affinity to Granzyme B. Such compounds may carry a radioactive moiety such as a radioisotope for therapeutic uses (therapeutic radioisotope). Such compounds have shown superior features, including good metabolism profiles, including little or no gut intake, and complete renal clearance with no production of metabolites. See International Patent Application No. PCT/US2022/081098, the relevant disclosures of which are incorporated by reference for the subject matter and purpose referenced herein.

Accordingly, provided herein are granzyme B-targeting theranostic therapies for treating cancer in subjects who need the treatment. A theranostic therapy comprises a granzyme B-targeting therapeutic compound comprising a therapeutic radioisotope as disclosed herein and optionally a granzyme B-targeting imaging compound. In some instances, the granzyme B-targeting therapeutic compound and the granzyme B-targeting imaging compound may be the same molecule loaded with different types of radioactive moieties, for example, a therapeutic radioisotope versus an imaging radioisotope. In some embodiments, the subject for treatment by the granzyme B-targeting theranostic therapy may have been undergone an immune therapy or is undergoing an immune therapy. In some embodiments, the theranostic therapy disclosed herein may be administered to the subject concurrently with an immune therapy.

### Definitions

It is to be understood that the terminology employed herein is for the purpose of describing particular embodiments and is not intended to be limiting. Further, although any methods, devices, and materials similar or equivalent to those described herein can be used in the practice or testing of the invention, the preferred methods, devices, and materials are now described. In addition to the foregoing, as used in the specification and appended claims, unless specified to the contrary, the following terms have the meaning indicated:
“Amino” refers to the —NH₂ radical.
“Cyano” refers to the —CN radical.
“Hydroxyl” refers to the —OH radical.
“Imino” refers to the ═NH substituent.
“Nitro” refers to the —NO₂ radical.
“Oxo” refers to the ═O substituent.
“Thioxo” refers to the ═S substituent.
“Trifluoromethyl” refers to the —CF₃ radical.
“Alkyl” refers to a linear, saturated, acyclic, monovalent hydrocarbon radical or branched, saturated, acyclic, monovalent hydrocarbon radical, having from one to six carbon atoms, and which is attached to the rest of the molecule by a single bond, e.g., methyl, ethyl, n-propyl, 1-methylethyl (iso-propyl), n-butyl, n-pentyl, 1,1-dimethylethyl (t-butyl), 3-methylpenty-1,2-methylpentyl and the like. An alkyl moiety may be unsubstituted. Alternatively, an alkyl moiety may be optionally substituted. An optionally substituted alkyl radical is an alkyl radical that is optionally substituted, valence permitting, by one, two, three, four, or five substituents independently selected from the group consisting of halo, cyano, nitro, aryl, cycloalkyl, heterocyclyl, heteroaryl, oxo, trimethylsilanyl, —OR³, —OC(O)—R³, —N(R³)₂, —C(O)R⁴, —C(O)OR₃, —C(O)N(R³)₂, —N(R³)C(O)OR⁵, —N(R³)C(O)R⁵, —N(R³)S(O)ₜR⁵ (where t is 1 or 2), —S(O)ₜOR⁵ (where t is 1 or 2), —S(O)ₚR⁵ (where p is 0, 1, or 2) and —S(O)ₜN(R³)₂ (where t is 1 or 2), where each R³ is independently hydrogen, alkyl, haloalkyl, cycloalkyl, cycloalkylalkyl, aryl, aralkyl, heterocyclyl, or heteroaryl; each R⁴ is independently hydrogen, cycloalkyl, aryl, heterocyclyl, or heteroaryl; and each R⁵ is independently alkyl, haloalkyl, cycloalkyl, aryl, heterocyclyl, or heteroaryl.

“Cycloalkyl” refers to a stable non-aromatic monocyclic or polycyclic hydrocarbon radical having from three to fifteen carbon atoms, preferably having from three to ten carbon atoms, and which is saturated or unsaturated, and which attaches to the rest of the molecule by a single bond. A polycyclic hydrocarbon radical is bicyclic, tricyclic, or tetracyclic ring system. An unsaturated cycloalkyl contains one, two, or three carbon-carbon double bonds and/or one carbon-carbon triple bond. Monocyclic cycloalkyl radicals include, for example, cyclopropyl, cyclobutyl, cyclopentyl, cyclohexyl, cycloheptyl, and cyclooctyl. Polycyclic cycloalkyl radicals include, for example, adamantyl, norbornyl, decalinyl, and the like. A cycloalkyl moiety may be unsubstituted. Alternatively, a cycloalkyl moiety may be optionally substituted. An optionally substituted cycloalkyl is a cycloalkyl radical that is optionally substituted by one, two, three, four, or five substituents independently selected from the group consisting of alkyl, alkenyl, halo, haloalkyl, haloalkenyl, cyano, nitro, oxo, aryl, aralkyl, cycloalkyl, heterocyclyl, heteroaryl, —R⁴—OR³, —R⁴—OC(O)—R³, 13 R⁴—N(R³)₂, —R⁴—C(O)R³, R⁴—C(O)OR³, —R⁴—C(O)N(R³)₂, —R⁴—N(R³)C(O)OR⁵, —R⁴—N(R³)C(O)R⁵, R⁴—N(R³)S(O),R⁵ (where t is 1 or 2), —R⁴—S(O)ₜOR⁵ (where t is 1 or 2), —R⁴—S(O)ₚR⁵ (where p is 0, 1, or 2) and —R⁴—S(O)ₜN(R³)₂ (where t is 1 or 2) where each R³ is independently hydrogen, alkyl, haloalkyl, cycloalkyl, aryl, heterocyclyl, or heteroaryl; each R⁴ is independently a direct bond or a linear or branched alkylene or alkenylene chain; and each R⁵ is independently alkyl, haloalkyl, cycloalkyl, cycloalkylalkyl, aryl, aralkyl, heterocyclyl, or heteroaryl.

“Chelating moieties” are those molecules or ions, which are able to act as a polydentate ligand to a metal ion. For example, molecules with multiple atoms with available lone pairs (including but not limited to nitrogen and oxygen) may act as chelating moieties. Chelating moieties may be linear (e.g., EDTA), or cyclic (including macrocycles e.g., DOTA, porphyrin) and may involve macrocyas commonly known in the art. Chelating moieties may have 2, 3, 4, 5, or 6 functional groups (e.g., amines, amides, hydroxyls, carboxylic acids etc.) with available lone pairs to coordinate with a metal.

In some embodiments, preparation of compounds can involve the addition of acids or bases to affect, for example, catalysis of a desired reaction or formation of salt forms such as acid addition salts.

Exemplary acids can be inorganic or organic acids and include, but are not limited to, strong and weak acids. Some example acids include hydrochloric acid, hydrobromic acid, sulfuric acid, phosphoric acid, p-toluenesulfonic acid, 4-nitrobenzoic acid, methanesulfonic acid, benzenesulfonic acid, trifluoroacetic acid, and nitric acid. Some weak acids include, but are not limited to acetic acid, propionic acid, butanoic acid, benzoic acid, tartaric acid, pentanoic acid, hexanoic acid, heptanoic acid, octanoic acid, nonanoic acid, and decanoic acid.

Exemplary bases include lithium hydroxide, sodium hydroxide, potassium hydroxide, lithium carbonate, sodium carbonate, potassium carbonate, and sodium bicarbonate. Some example strong bases include, but are not limited to, hydroxide, alkoxides, metal amides, metal hydrides, metal dialkylamides and arylamines, wherein; alkoxides include lithium, sodium and potassium salts of methyl, ethyl and t-butyl oxides; metal amides include sodium amide, potassium amide and lithium amide; metal hydrides include sodium hydride, potassium hydride and lithium hydride; and metal dialkylamides include lithium, sodium, and potassium salts of methyl, ethyl, n-propyl, iso-propyl, n-butyl, tert-butyl, trimethylsilyl and cyclohexyl substituted amides.

As used herein, the phrase “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present application include the conventional non-toxic salts of the parent compound formed, for example, from non-toxic inorganic or organic acids. The pharmaceutically acceptable salts of the present application can be synthesized from the parent compound, which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, non-aqueous media like ether, ethyl acetate, alcohols (e.g., methanol, ethanol, iso-propanol, or butanol) or acetonitrile (MeCN) are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977). Conventional methods for preparing salt forms are described, for example, in Handbook of Pharmaceutical Salts: Properties, Selection, and Use, Wiley-VCH, 2002.

In some embodiments, the compounds provided herein, or salts thereof, are substantially isolated. By “substantially isolated” is meant that the compound is at least partially or substantially separated from the environment in which it was formed or detected. Partial separation can include, for example, a composition enriched in the compounds provided herein. Substantial separation can include compositions containing at least about 50%, at least about 60%, at least about 70%, at least about 80%, at least about 90%, at least about 95%, at least about 97%, or at least about 99% by weight of the compounds provided herein, or salt thereof. Methods for isolating compounds and their salts are routine in the art.

The expressions “ambient temperature” and “room temperature” or “rt” as used herein, are understood in the art, and refer generally to a temperature, e.g. a reaction temperature, that is about the temperature of the room in which the reaction is carried out, for example, a temperature from about 20° C. to about 30° C.

### I. Granzyme B-Targeting Compounds

In some aspects, provided herein are Granzyme B-targeting compounds disclosed herein, e.g., Formula (I) compounds. The compounds disclosed herein encompass the compounds per se, their pharmaceutically acceptable salt thereof, and stereoisomers thereof.

Compounds described herein can comprise one or more asymmetric centers, and thus can exist in various isomeric forms, e.g., enantiomers and/or diastereomers. For example, the compounds described herein can be in the form of an individual enantiomer, diastereomer or geometric isomer, or can be in the form of a mixture of stereoisomers, including racemic mixtures and mixtures enriched in one or more stereoisomer. Isomers can be isolated from mixtures by methods known to those skilled in the art, including chiral high-performance liquid chromatography (HPLC) and the formation and crystallization of chiral salts; or preferred isomers can be prepared by asymmetric syntheses. See, for example, Jacques et al., Enantiomers, Racemates and Resolutions (Wiley Interscience, New York, 1981); Wilen et al., Tetrahedron 33:2725 (1977); Eliel, Stereochemistry of Carbon Compounds (McGraw-Hill, NY, 1962); and Wilen, Tables of Resolving Agents and Optical Resolutions p. 268 (E .L. Eliel, Ed., Univ. of Notre Dame Press, Notre Dame, IN 1972). The disclosure additionally encompasses compounds described herein as individual isomers substantially free of other isomers, and alternatively, as mixtures of various isomers.

### A. Compounds of Formula (I)

The GZB-targeting compounds provided herein may have the structure of Formula (I):

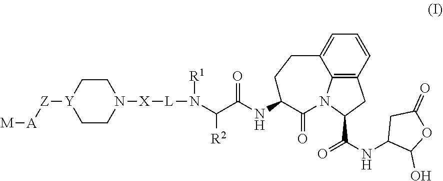

or a stereoisomer, tautomer, or a salt thereof. In Formula (I) M is a radioactive moiety; A is a chelating moiety chelating the radioactive moiety; X is —CH₂C(NH)—, —CH₂C(O)—, —CH₂C(S)—, —NHC(NH)—, —NHC(O)—, —NHC(S)—, —OC(NH)—, —OC(O)—, or —OC(S)—; Y is CH or N; Z is —CH₂—, —CH₂C(NH)—, —CH₂C(O)—, —CH₂C(S)—, —NHC(NH)—, —NHC(O)—, —NHC(S)—, —OC(NH)—, —OC(O)—, and —OC(S)—; L is a peptide linker having 1-6 amino acid residues, inclusive; R¹ is H or C₁₋₆ alkyl; and R² is C₁₋₆ alkyl or C₃₋₆ cycloalkyl.

In some embodiments, R¹ is H. In other embodiments, R¹ is C₁₋₆ alkyl. In other embodiments, R¹ is C₁ alkyl. In other embodiments, R¹ is C₂ alkyl. In other embodiments, R¹ is C₃ alkyl. In other embodiments, R¹ is C₄ alkyl. In other embodiments, R¹ is C₅ alkyl. In other embodiments, R¹ is C₆ alkyl. In some certain embodiments, R¹ is a methyl ( —CH₃).

In some embodiments, R² is C₁₋₆ alkyl. In some certain embodiments, R² is C₃₋₆ alkyl. For example, C₃₋₆ alkyl of R² is branched or unbranched, substituted or unsubstituted C₃₋₆ alkyl. In some embodiments, R² is C₃ alkyl. In some embodiments, R² is C₄ alkyl. In some embodiments, R² is Cs alkyl. In some embodiments, R² is C₆ alkyl. In some embodiments, R² is sec-butyl (—CH(CH₃)CH₂CH₃).

In some embodiments, R² is C₃₋₆ cycloalkyl. In some embodiments, C₃₋₆ cycloalkyl of R² is cyclopropyl, cyclobutyl, cyclopentyl, or cyclohexyl.

In one embodiment, the compound has a structure of Formula (Ia):

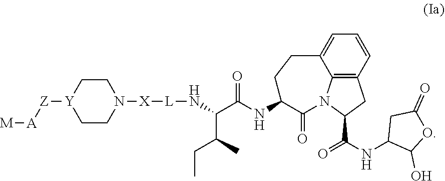

In another embodiment, the compound has a structure of Formula (Ib):

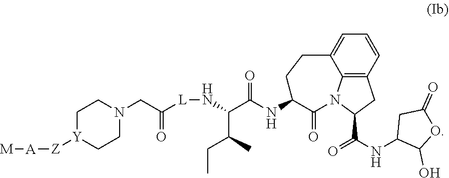

In some examples, X is —CH₂C(NH)—. In some examples, X is —CH₂C(O)—. In some examples, X is —CH₂C(S)—. In some examples, X is —NHC(NH)—. In some examples, X is —NHC(O)—. In some examples, X is —NHC(S)—. In some examples, X is —OC(NH)—. In some examples, X is —OC(O)—. In some examples, X is —OC(S)—. In specific examples, X is —CH₂C(O)—.

In some examples, Z is —CH₂—. In some examples, Z is —CH₂C(NH)—. In some examples, Z is —CH₂C(O)—. In some examples, Z is —CH₂C(S)—. In some examples, Z is —NHC(NH)—. In some examples, Z is —NHC(O)—. In some examples, Z is —NHC(S)—. In some examples, Z is —OC(NH)—. In some examples, Z is —OC(O)—. In some examples, Z is —OC(S)—.

In any of the Formula (I) compounds, e.g., Formula (Ia) and Formula 9Ib) compounds, L is a peptide linker having 1-6 amino acid residues, inclusive. In some embodiments, L has 1-5 amino acid residues, inclusive. In some embodiments, L has 2-4 amino acid residues, inclusive. In some examples, L has 1 amino acid residue. In some examples, L has 2 amino acid residues. In some examples, L has 3 amino acid residues. In some examples, L has 4 amino acid residues. In some examples, L has 5 amino acid residues. In some examples, L has 6 amino acid residues.

In some embodiments, the amino acid residues in any of the L peptides are standard proteinogenic amino acids (i.e., the 20 naturally-occurring amino acid residues found in naturally-occurring proteins), or unnatural amino acids, which may be derivatives of a natural-occurring protein or an isomer of a naturally-occurring amino acid residue. As used herein, proteinogenic amino acid residues refer to the 20 amino acid residues existing in nature as building blocks for synthesizing proteins. Amino acid residues may form a chain through standard peptide bonds, or by forming amide bonds with compatible side chains (e.g., glutamic acid (e.g., D-Glu), aspartic acid). Structures of exemplary non-naturally occurring amino acid residues that may be included in the L linker are provided in Table 1 below.

TABLE 1
Exemplary Non-Proteinogenic Amino Acids

| Chemical Structure | Non-Naturally Occurring Amino Acids |
|---|---|
| 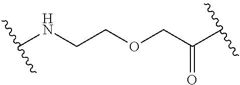 [SMILES: *NCCOCC(*)=O] | AEA |
| 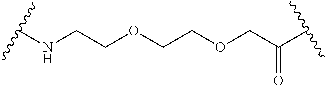 [SMILES: *NCCOCCOCC(*)=O] | AEEA |
| 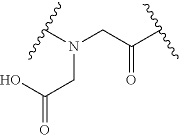 [SMILES: *C(=O)CN(*)CC(=O)O] | N-acid |
| 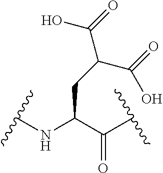 [SMILES: *N[C@@H](CC(C(=O)O)C(=O)O)C(*)=O] | Diacid |
| 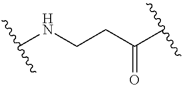 [SMILES: *NCCC(*)=O] | βAla |
| 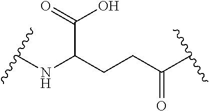 [SMILES: *NC(CCC(*)=O)C(=O)O] | γGlu |

Exemplary amino acid sequences for the L peptide include Glu-Gly-Gly, Glu-βAla-βAla, γGlu, _{D}γGlu, γGlu-βAla, _{D}Glu-βAla-βAla, _{D}Glu-AEA, _{D}Glu-AEEA-AEEA, _{D}Glu-_{D}Glu-AEA, _{D}Glu-DGlu-βAla-βAla, βAla-_{D}Glu-βAla, Diacid-βAla-βAla, N-acid-βAla-βAla, and βAla-N-acid-βAla. In some embodiments, L has the sequence of Glu-Gly-Gly. In some embodiments, L has the sequence of Glu-βAla-βAla. In some embodiments, L has the sequence of γGlu. In some embodiments, L has the sequence of _{D}γGlu. In some embodiments, L has the sequence of γGlu-βAla. In some embodiments, L has the sequence of _{D}Glu-βAla-βAla. In some embodiments, L has the sequence of _{D}Glu-AEA. In some embodiments, L has the sequence of _{D}Glu-AEEA-AEEA. In some embodiments, L has the sequence of _{D}Glu-_{D}Glu-AEA. In some embodiments, L has the sequence of _{D}Glu-_{D}Glu-βAla-βAla. In some embodiments, L has the sequence of βAla-_{D}Glu-βAla. In some embodiments, L has the sequence of Diacid-BAla-BAla. In some embodiments, L has the sequence of N-acid-βAla-βAla. In some embodiments, L has the sequence of βAla-N-acid-βAla. See Table 2 for structures of these exemplary L linkers.

TABLE 2
A list of Peptide Linkers

| Chemical Structure | L Sequence |
|---|---|
| 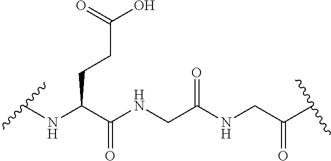 [SMILES: *N[C@@H](CCC(=O)O)C(=O)NCC(=O)NCC(*)=O] | Glu-Gly-Gly |
| 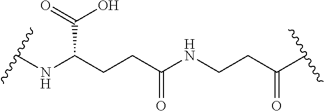 [SMILES: *N[C@@H](CCC(=O)NCCC(*)=O)C(=O)O] | γGlu-βAla |
| 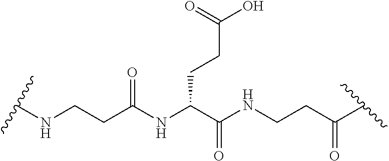 [SMILES: *NCCC(=O)N[C@H](CCC(=O)O)C(=O)NCCC(*)=O] | βAla-_{D}Glu-βAla |
| 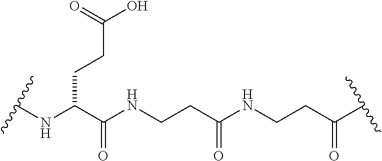 [SMILES: *N[C@H](CCC(=O)O)C(=O)NCCC(=O)NCCC(*)=O] | Glu-βAla-βAla |
| 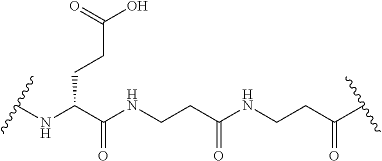 [SMILES: *N[C@H](CCC(=O)O)C(=O)NCCC(=O)NCCC(*)=O] | _{D}Glu-βAla-βAla |
| 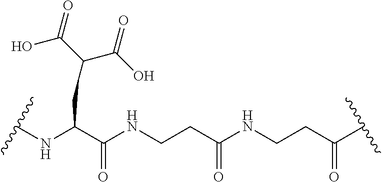 [SMILES: *N[C@@H](CC(C(=O)O)C(=O)O)C(=O)NCCC(=O)NCCC(*)=O] | Diacid-βAla-βAla |
| 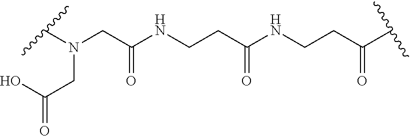 [SMILES: *C(=O)CCNC(=O)CCNC(=O)CN(*)CC(=O)O] | N-acid-βAla-βAla |
| 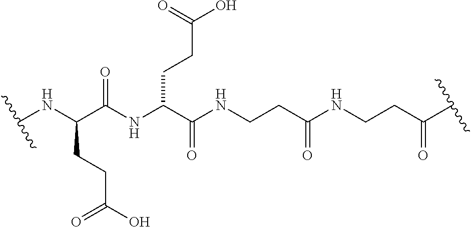 [SMILES: *N[C@H](CCC(=O)O)C(=O)N[C@H](CCC(=O)O)C(=O)NCCC(=O)NCCC(*)=O] | _{D}Glu-_{D}Glu-βAla-βAla |
| 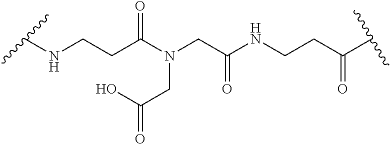 [SMILES: *NCCC(=O)N(CC(=O)O)CC(=O)NCCC(*)=O] | βAla-N-acid-βAla |
| 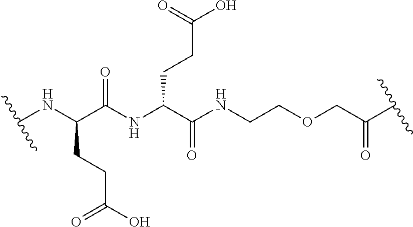 [SMILES: *N[C@H](CCC(=O)O)C(=O)N[C@H](CCC(=O)O)C(=O)NCCOCC(*)=O] | _{D}Glu-_{D}Glu-AEA |
| 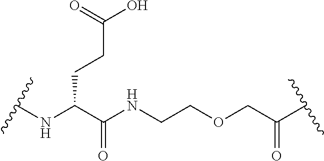 [SMILES: *N[C@H](CCC(=O)O)C(=O)NCCOCC(*)=O] | _{D}Glu-AEA |
| 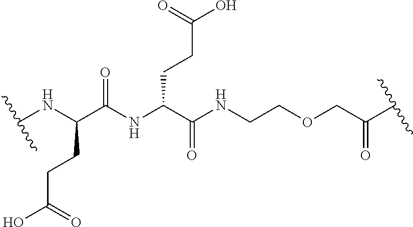 [SMILES: *N[C@H](CCC(=O)O)C(=O)N[C@H](CCC(=O)O)C(=O)NCCOCC(*)=O] | _{D}Glu-_{D}Glu-AEA |
| 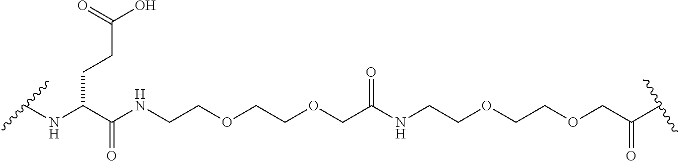 [SMILES: *N[C@H](CCC(=O)O)C(=O)NCCOCCOCC(=O)NCCOCCOCC(*)=O] | _{D}Glu-AEEA-AEEA |
| 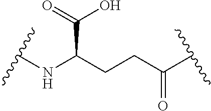 [SMILES: *N[C@H](CCC(*)=O)C(=O)O] | γGlu |
| 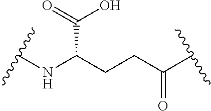 [SMILES: *N[C@@H](CCC(*)=O)C(=O)O] | _{D}γGlu |

In some embodiments, Y is CH, forming a piperidine ring. In other embodiments, Y is N forming a piperazine ring.

In some specific examples, Z is —CH₂—. In other specific examples, Z is —CH₂C(O)—.

In some embodiments, the compound of Formula (I) provided herein may have one of the following structures of Formula (Ib-A) or (Ib-B) (variables A, L, and M are as defined herein):

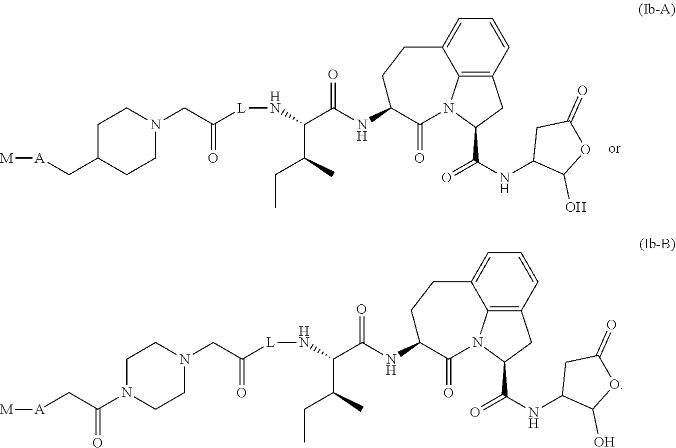

In Formula (I), A is a chelating moiety. Chelating moieties are those molecules or ions, which are able to act as a polydentate ligand to a metal ion. For example, molecules with multiple atoms with available lone pairs (including but not limited to nitrogen and oxygen) may act as chelating moieties. Chelating moieties may be linear (e.g., EDTA), or cyclic (including macrocycles e.g., DOTA, porphyrin) and may involve macrocyas commonly known in the art. Chelating moieties may have 2, 3, 4, 5, or 6 functional groups (e.g., amines, amides, hydroxyls, carboxylic acids etc.) with available lone pairs to coordinate with a metal. Exemplary chelating moieties for use in the Granzyme B-targeting compounds disclosed herein include, but are not limited to, 1,4,7-triazacyclononanetriacetic acid (NOTA), 2-S-(4-isothiocyanatobenzyl)-1,4,7-triazacyclononane-1,4,7-triacetic acid (p-SCN-Bm-NOTA), 1,4,7,10-tetraazacyclododecane-1,4,7,10-tetraacetic acid (DOTA), 1,4,7-triazacyclononane-1-glutaric acid-4,7-diacetic acid (NODAGA), ethylene diamine tetra-acetic acid (EDTA), diethylene triaminepentaacetic acid (DTPA), cyclohexyl-1,2-diaminetetraacetic acid (CDTA), ethyleneglycol-0,0′-bis (2-aminoethyl)-N,N,N′,N′-tetraacetic acid (EGTA), N,N-bis (hydroxybenzyl)-ethylenediamine-N,N′-diacetic acid (HBED), triethylene tetramine hexaacetic acid (TTHA), hydroxyethyidiamine triacetic acid (HEDTA), 1,4,8,11-tetraazacyclotetradecane-N,N′, N″,N″′-tetraacetic acid (TETA), 1,4,7,10-tetraaza-1,4,7,10-tetra-(2-carbamoyl methyl)-cyclododecane (TCMC), 1,4,7-triazacyclononane-4,7-diyl diacetic acid (NODA) and Desferrioxamine B (DFO). In some embodiments, the chelating agent is selected from the group consisting of 1,4,7-triazacyclononanetriacetic acid (NOTA), 1,4,7,10-tetraazacyclododecane-1,4,7,10-tetraacetic acid (DOTA), 1,4,7-triazacyclononane-4,7-diyl diacetic acid (NODA), 1,4,7-triazacyclononane-l-glutaric acid-4,7-diacetic acid (NODAGA), Restrained Complexing Agent (RESCA), and MACROPA. In some embodiments, the chelating agent is 1,4,7-triazacyclononanetriacetic acid (NOTA). In other embodiments, the chelating agent is 1,4,7,10-tetraazacyclododecane-1,4,7,10-tetraacetic acid (DOTA).

In one embodiment, the chelating moiety A is 1,4,7-triazacyclononane-N,N′,N′-triacetic acid (NOTA) or 1,4,7-triazacyclononane-4,7-diyl diacetic acid (NODA). In some embodiments, the chelating moiety A is 1,4,7-triazacyclononane-N,N′,N″-triacetic acid (NOTA). In some embodiments, 1,4,7-triazacyclononane-N,N′,N″-triacetic acid (NOTA) of A has the following structure:

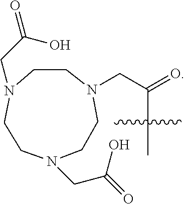 [SMILES: *C(=O)CN1CCN(CC(=O)O)CCN(CC(=O)O)CC1]

In some embodiments, the chelating moiety A is 1,4,7-triazacyclononane-4,7-diyl diacetic acid (NODA). In some embodiments, 1,4,7-triazacyclononane-4,7-diyl diacetic acid (NODA) of A has the following structure:

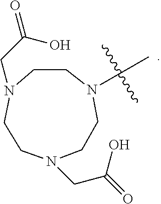 [SMILES: *N1CCN(CC(=O)O)CCN(CC(=O)O)CC1]

In some embodiments, the chelating moiety A is 1,4,7,10-tetraazacyclododecane-1,4,7,10-tetraacetic acid (DOTA), which has the following structure:

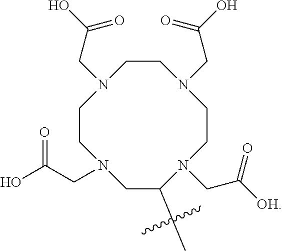 [SMILES: *C1CN(CC(=O)O)CCN(CC(=O)O)CCN(CC(=O)O)CCN1CC(=O)O]

In some embodiments, the chelating moiety A is 1,4,7,10-Tetraazacyclododecane-1,4,7-triacetic acid (DO3A), which has the following structure:

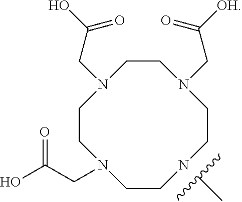

Selection of suitable chelating agents in pair with therapeutic radioisotopes may follow conventional methods (see, e.g., Sgouros et al., Nature Reviews, 19:589-608, 2020 and Poty et al., J. Nuclear Medicine, 59 (6): 878-884; 2018, the relevant disclosures of each of which are incorporated by reference for the subject matter and purpose referenced herein), or the guidance provided herein. In some instances, a compound of Formula (I) may have the pair of chelating agent and therapeutic radioisotopes listed in Table 3 below. Such pairs of chelating agents and therapeutic radioisotopes could result in high loading efficiency of the therapeutic radioisotopes.

TABLE 3
Chelating Agents and Therapeutic Radioisotopes

| Chelating Agent | Therapeutic Radioisotopes |
|---|---|
| NODA | ⁴⁷Sc or ⁶⁷Cu |
| NOTA | ⁹⁰Y, ¹⁷⁷Lu, or ²²⁵Ac. |
| DOTA | ⁹⁰Y, ¹⁷⁷Lu, or ²²⁵Ac. |

In some embodiments, the radioactive moiety of M in any of the Formula (I) compounds disclosed herein (e.g., a compound of Formula (Ia) or a compound of Formula (Ib) such as Formula (Ib-A) or Formula (Ib-B) can be a therapeutic radioisotope. In some embodiments, the therapeutic radioisotope of M is ⁶⁷Cu. In some embodiments, the therapeutic radioisotope of M is ⁹⁰Y. In some embodiments, the therapeutic radioisotope of M is ¹⁷⁷Lu. In some embodiments, the therapeutic radioisotope of M is ²²⁵Ac. In some embodiments, the therapeutic radioisotope of M is ⁴⁷Sc. In some embodiments, the therapeutic radioisotope of M is ¹³¹I. In some embodiments, the therapeutic radioisotope of M is ¹⁵³Sm. In some embodiments, the therapeutic radioisotope of M is ¹⁶¹Tb. In some embodiments, the therapeutic radioisotope of M is ²¹¹At. In some embodiments, the therapeutic radioisotope of M is 212Pb. In some embodiments, the therapeutic radioisotope of M is ²¹²Bi. In some embodiments, the therapeutic radioisotope of M is ²²³Ra. In some embodiments, the therapeutic radioisotope of M is ²²⁷mh. In some certain embodiments, the chelating moiety is NOTA or DOTA, and wherein the therapeutic radioisotope is ⁹⁰Y, ¹⁷⁷Lu, or ²²⁵Ac. In some other embodiments, the chelating moiety is NODA, and the therapeutic radioisotope is ⁴⁷Sc or ⁶⁷Cu.

Exemplary Formula (I) compounds are provided in Table 4 below.

TABLE 4
Exemplary Compounds of Formula (Ib-A)

| Compounds | ID |
|---|---|
| 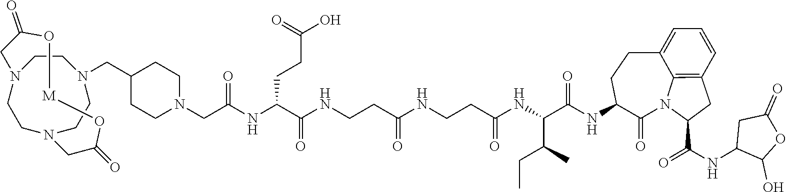 | 1-M |
| wherein M is ¹⁷⁷Lu, ⁹⁰Y, ²²⁵Ac, or ²¹³Bi | |
| 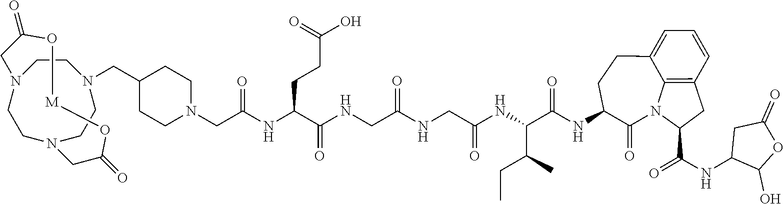 | 2-M (SEQ ID NO: 1) |
| wherein M is ¹⁷⁷Lu, ⁹⁰Y, ²²⁵Ac, or ²¹³Bi | |
| 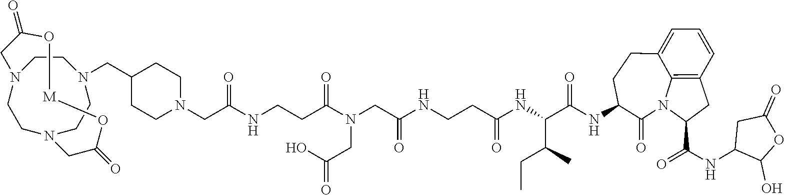 | 3-M |
| wherein M is ¹⁷⁷Lu, ⁹⁰Y, ²²⁵Ac, or ²¹³Bi | |
| 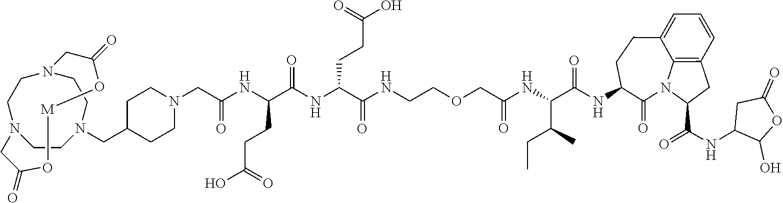 | 4-M |
| wherein M is ¹⁷⁷Lu, ⁹⁰Y, ²²⁵Ac, or ²¹³Bi | |
| 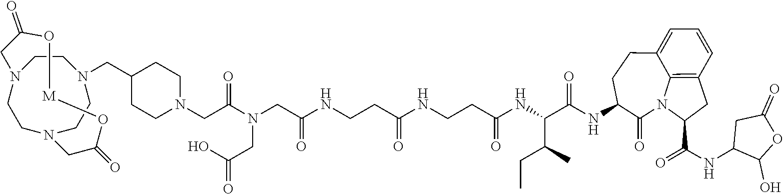 | 5-M |
| wherein M is ¹⁷⁷Lu, ⁹⁰Y, ²²⁵Ac, or ²¹³Bi | |
| 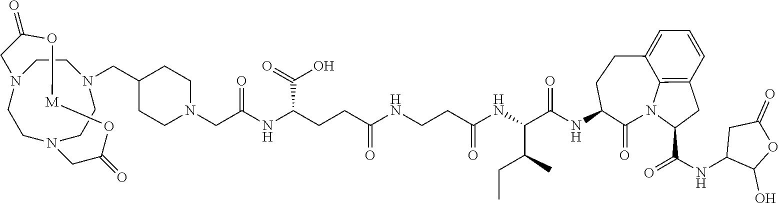 | 6-M |
| wherein M is ¹⁷⁷Lu, ⁹⁰Y, ²²⁵Ac, or ²¹³Bi | |
| 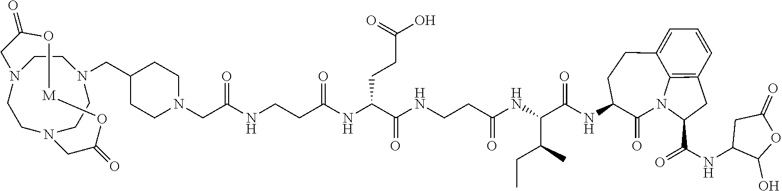 | 7-M |
| wherein M is ¹⁷⁷Lu, ⁹⁰Y, ²²⁵Ac, or ²¹³Bi | |

The present application also includes stereoisomers of the compounds, such as stereoisomers of Compounds 1-M and 4-M described herein and disclosed in Table 5 below. The stereoisomers result from the two chiral centers (closed ring) or one chiral center (open chain) of the hemiacetal unit as indicated by the wiggle lines:

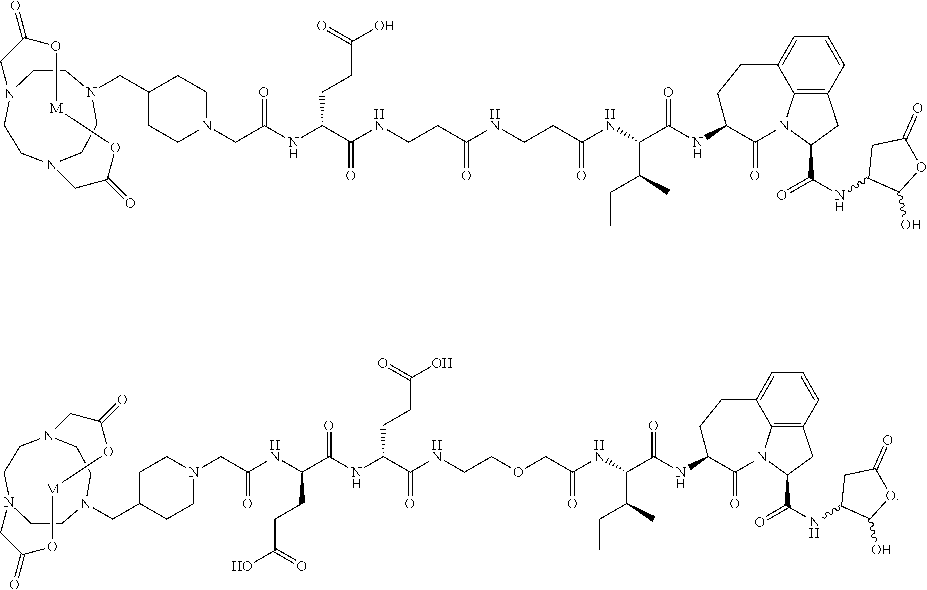

TABLE 5
Stereoisomers of Compounds 1-M and 4-M

| Compounds | ID |
|---|---|
| 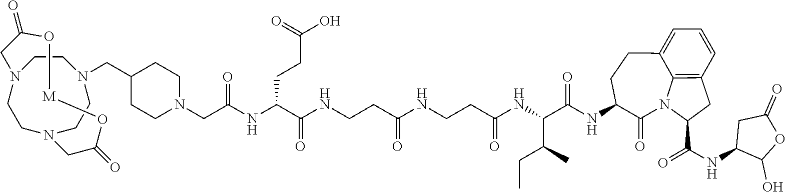 | 1-M (S) |
| wherein M is ¹⁷⁷Lu, ⁹⁰Y, ²²⁵Ac, or ²¹³Bi | |
| 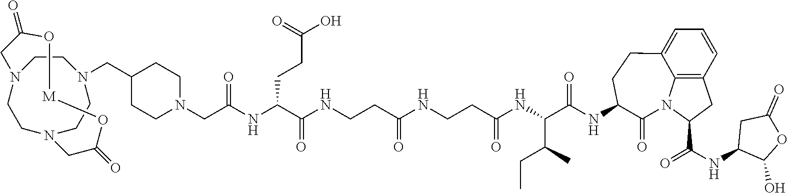 | 1-M (S)(S) |
| wherein M is ¹⁷⁷Lu, ⁹⁰Y, ²²⁵Ac, or ²¹³Bi | |
| 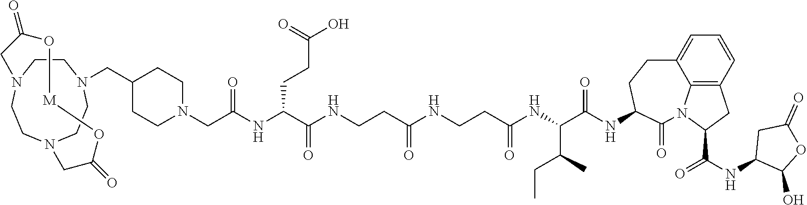 | 1-M (S)(R) |
| wherein M is ¹⁷⁷Lu, ⁹⁰Y, ²²⁵Ac, or ²¹³Bi | |
| 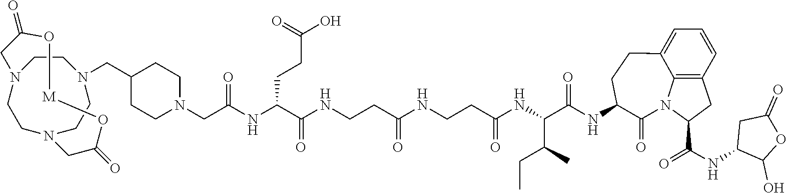 | 1-M (R) |
| wherein M is ¹⁷⁷Lu, ⁹⁰Y, ²²⁵Ac, or ²¹³Bi | |
| 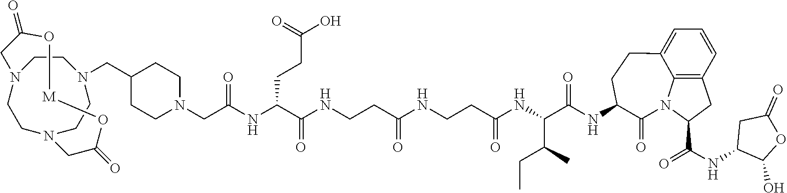 | 1-M (R)(S) |
| wherein M is ¹⁷⁷Lu, ⁹⁰Y, ²²⁵Ac, or ²¹³Bi | |
| 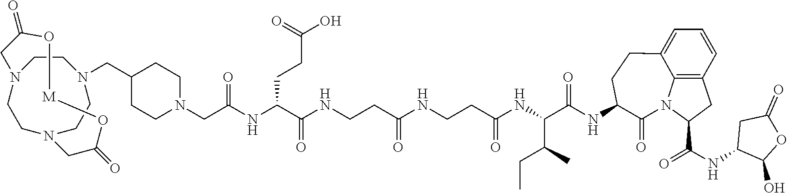 | 1-M (R)(R) |
| wherein M is ¹⁷⁷Lu, ⁹⁰Y, ²²⁵Ac, or ²¹³Bi | |
| 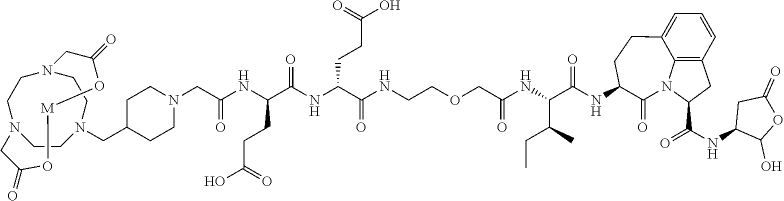 | 4-M (S) |
| wherein M is ¹⁷⁷Lu, ⁹⁰Y, ²²⁵Ac, or ²¹³Bi | |
| 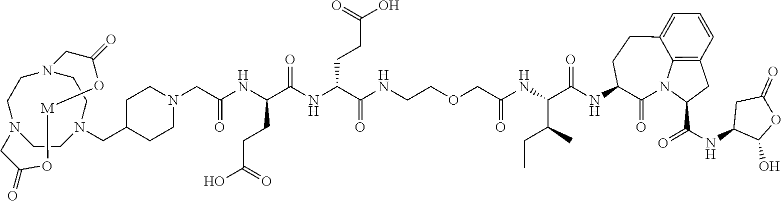 | 4-M (S)(S) |
| wherein M is ¹⁷⁷Lu, ⁹⁰Y, ²²⁵Ac, or ²¹³Bi | |
| 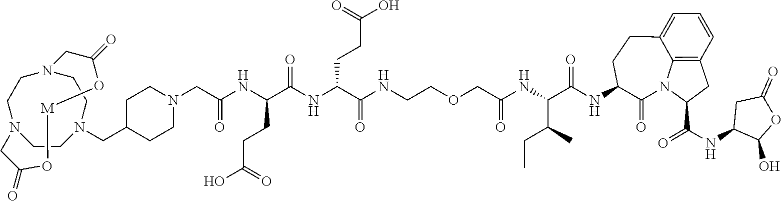 | 4-M (S)(R) |
| wherein M is ¹⁷⁷Lu, ⁹⁰Y, ²²⁵Ac, or ²¹³Bi | |
| 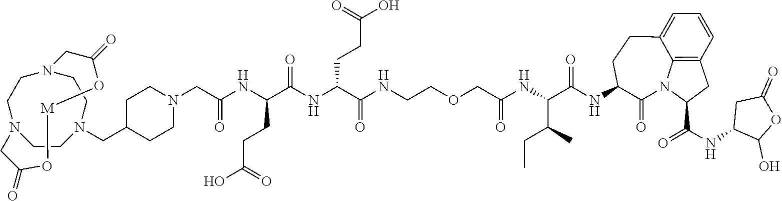 | 4-M (R) |
| wherein M is ¹⁷⁷Lu, ⁹⁰Y, ²²⁵Ac, or ²¹³Bi | |
| 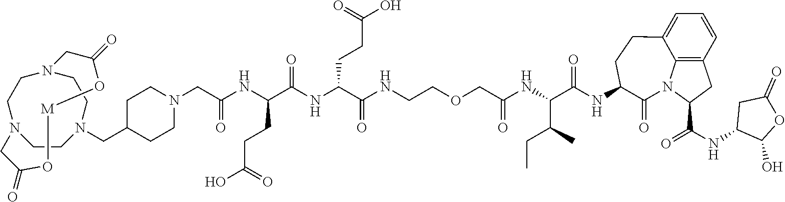 | 4-M (R)(S) |
| wherein M is ¹⁷⁷Lu, ⁹⁰Y, ²²⁵Ac, or ²¹³Bi | |
| 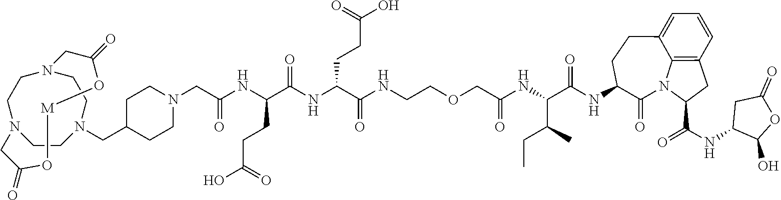 | 4-M (R)(R) |
| wherein M is ¹⁷⁷Lu, ⁹⁰Y, ²²⁵Ac, or ²¹³Bi | |

Exemplary compounds of Formula Ib-Bare disclosed in Table 6.

TABLE 6
Exemplary Compounds of Formula (Ib-B)

| Compounds | ID |
|---|---|
| 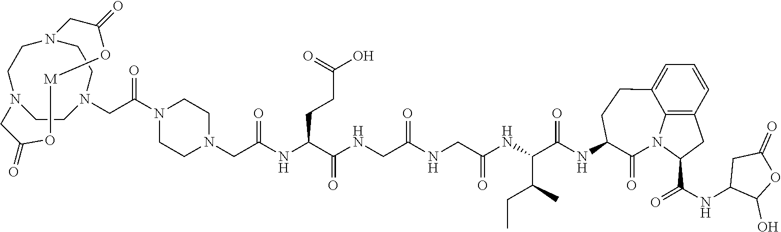 | 8-M (SEQ ID NO: 2) |
| wherein M is ¹⁷⁷Lu, ⁹⁰Y, ²²⁵Ac, or ²¹³Bi | |
| 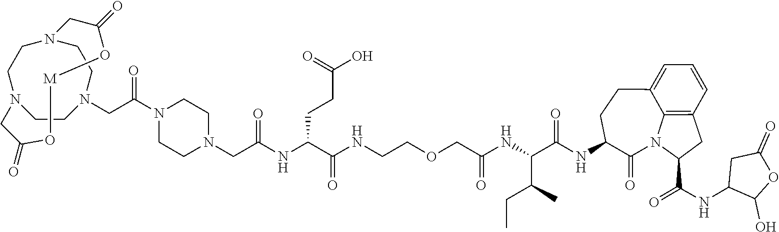 | 9-M |
| wherein M is ¹⁷⁷Lu, ⁹⁰Y, ²²⁵Ac, or ²¹³Bi | |
| 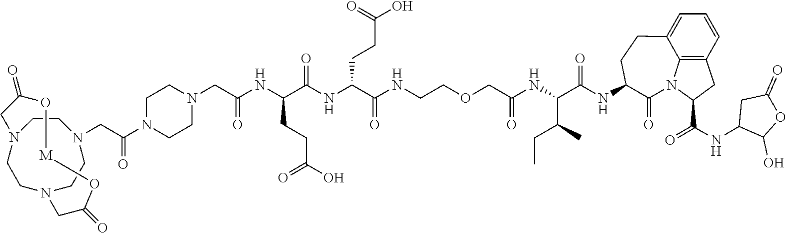 | 10-M |
| wherein M is ¹⁷⁷Lu, ⁹⁰Y, ²²⁵Ac, or ²¹³Bi | |
| 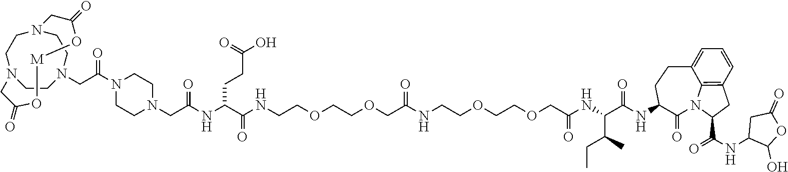 | 11-M |
| wherein M is ¹⁷⁷Lu, ⁹⁰Y, ²²⁵Ac, or ²¹³Bi | |
| 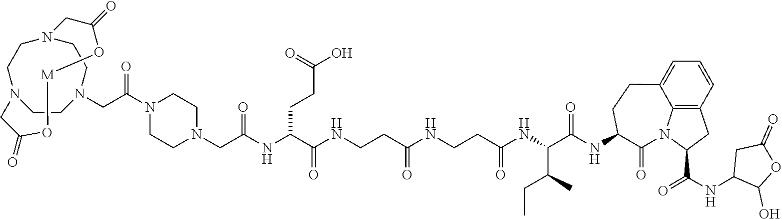 | 12-M |
| wherein M is ¹⁷⁷Lu, ⁹⁰Y, ²²⁵Ac, or ²¹³Bi | |
| 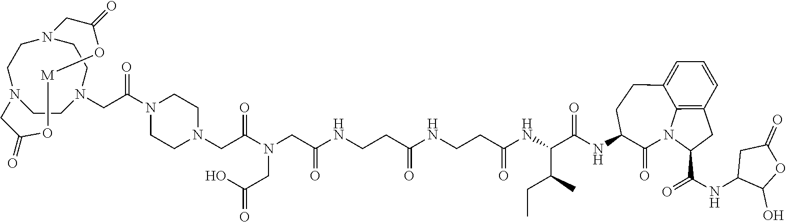 | 13-M |
| wherein M is ¹⁷⁷Lu, ⁹⁰Y, ²²⁵Ac, or ²¹³Bi | |
| 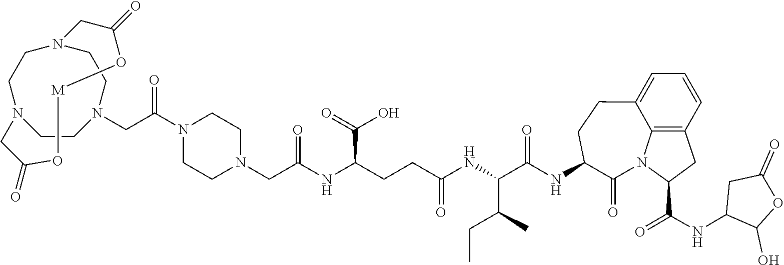 | 14-M |
| wherein M is ¹⁷⁷Lu, ⁹⁰Y, ²²⁵Ac, or ²¹³Bi | |
| 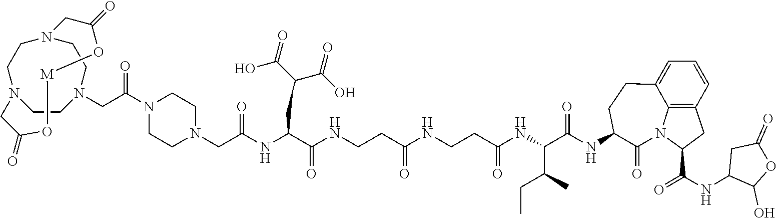 | 15-M |
| wherein M is ¹⁷⁷Lu, ⁹⁰Y, ²²⁵Ac, or ²¹³Bi | |
| 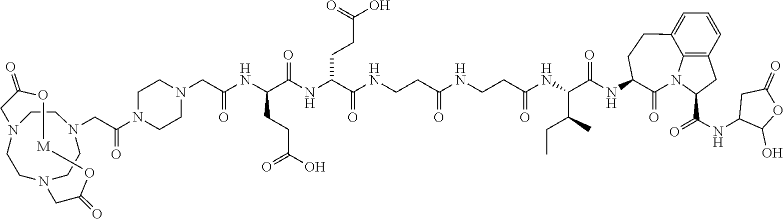 | 16-M |
| wherein M is ¹⁷⁷Lu, ⁹⁰Y, ²²⁵Ac, or ²¹³Bi | |
| 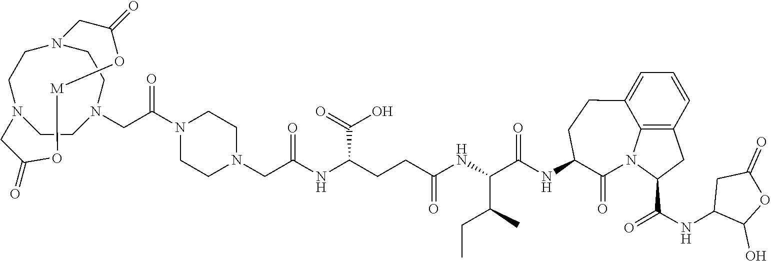 | 17-M |
| wherein M is ¹⁷⁷Lu, ⁹⁰Y, ²²⁵Ac, or ²¹³Bi | |
| 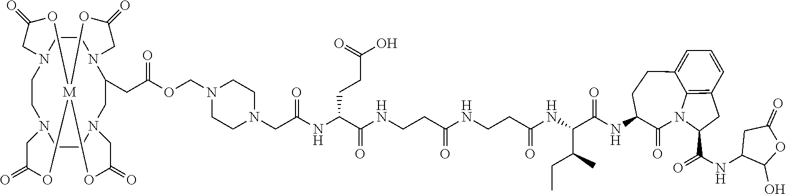 | 18-M |
| wherein M is ¹⁷⁷Lu, ⁹⁰Y, ²²⁵Ac, or ²¹³Bi | |

As shown in Examples below, the GZB-binding compounds disclosed herein, comprising either piperidine or piperazine rings linking the peptide linker and the chelating moiety, as well as specific peptide linker structures, showed better in vivo binding to GZB and clearance profiles as compared with other GZB-binding compounds. See International Application No.: PCT/US2022/081125 and WO2021/252644, and the relevant disclosures of which are incorporated by reference for the subject matter and purpose referenced herein.

Exemplary improved properties include improved pharmacokinetics (e.g., renal clearance), pharmacodynamics, and efficacy. In particular, the improved pharmacokinetics can be seen in the absence of gut intake, the absence of radiometabolites in urine, and/or predominant renal clearance. See International Application No.: PCT/US2022/081125.

The above compounds, when containing a radioisotope such as a therapeutic radioisotope, are useful as theranostic agents in one or more of the methods provided herein for treating GZB-related diseases such as cancer. As pointed out above, the present application also includes pharmaceutically acceptable salts of the compounds described herein. The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

### B. Chemical Synthesis of Granzyme B-Targeting Compounds

As will be appreciated, the compounds provided herein, including stereoisomers and salts thereof, can be prepared using known organic synthesis techniques and can be synthesized according to any of numerous possible synthetic routes.

The compounds disclosed herein, or a pharmaceutically acceptable salt thereof, can be prepared by following the exemplary protocols described below. Appropriate protective groups for use in such syntheses are known in the field. See, e.g., McOmic, Protective Groups in Organic Chemistry, (1973):98.

General synthetic procedures, and working examples thereof, for the preparation of peptide linker L, the Fmoc-Haic (2S, 5S)—OH tricycle moiety, and the appropriate metal complexation with the chelating moiety, can be found in International Application No.: PCT/US2021/036661, filed on Jun. 9, 2021, the relevant disclosures of which are incorporated by reference for the subject matter and purpose referenced herein.

Many appropriate radioactive moieties (e.g., radioisotopes such as therapeutic radioisotopes, e.g., those disclosed herein) are known in the art (see, for e.g., U.S. Pat. Nos. 5,021,236; 4,938,948; and 4,472,509, the disclosure of each of which is incorporated herein by reference in its entirety). Radioactively conjugated compounds, or a pharmaceutically acceptable salt thereof, provided herein may be prepared according to well-known methods in the art. Synthetic methods for incorporating radioisotopes into organic compounds are well known in the art, and one of ordinary skill in the art will readily recognize other methods applicable for the compounds provided herein.

It will be appreciated by one skilled in the art that the processes described herein are not the exclusive means by which compounds provided herein may be synthesized and that a broad repertoire of synthetic organic reactions is available to be potentially employed in synthesizing compounds provided herein. The person skilled in the art knows how to select and implement appropriate synthetic routes. Suitable synthetic methods of starting materials, intermediates and products may be identified by reference to the literature, including reference sources such as: Advances in Heterocyclic Chemistry, Vols. 1-107 (Elsevier, 1963-2012); Journal of Heterocyclic Chemistry Vols. 1-49 (Journal of Heterocyclic Chemistry, 1964-2012); Carreira, et al. (Ed.) Science of Synthesis, Vols. 1-48 (2001-2010) and Knowledge Updates KU2010/1-4; 2011/1-4; 2012/1-2 (Thieme, 2001-2012); Katritzky, et al. (Ed.) Comprehensive Organic Functional Group Transformations, (Pergamon Press, 1996); Katritzky et al. (Ed.); Comprehensive Organic Functional Group Transformations II(Elsevier, 2^{nd} Edition, 2004); Katritzky et al. (Ed.), Comprehensive Heterocyclic Chemistry (Pergamon Press, 1984); Katritzky et al., Comprehensive Heterocyclic Chemistry II, (Pergamon Press, 1996); Smith et al., March's Advanced Organic Chemistry: Reactions, Mechanisms, and Structure, 6^{th} Ed. (Wiley, 2007); Trost et al. (Ed.), Comprehensive Organic Synthesis (Pergamon Press, 1991).

The reactions for preparing compounds described herein can be carried out in suitable solvents which can be readily selected by one of skill in the art of organic synthesis. Suitable solvents can be substantially non-reactive with the starting materials (reactants), the intermediates, or products at the temperatures at which the reactions are carried out, (e.g., temperatures which can range from the solvent's freezing temperature to the solvent's boiling temperature). A given reaction can be carried out in one solvent or a mixture of more than one solvent. Depending on the particular reaction step, suitable solvents for a particular reaction step can be selected by the skilled artisan.

Preparation of the compounds described herein can involve the protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999).

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC), liquid chromatography-mass spectroscopy (LCMS), or thin layer chromatography (TLC). Compounds can be purified by those skilled in the art by a variety of methods, including high performance liquid chromatography (HPLC) and normal phase silica chromatography.

### II. Pharmaceutical Compositions

Any of the compounds of Formula (I), e.g., compounds of Formula (Ia) or compounds of Formula (Ib) such as Formula (Ib-A) and Formula (Ib-B), or a pharmaceutically acceptable salt thereof, may be mixed with a pharmaceutically acceptable carrier to form a pharmaceutical composition for, e.g., therapeutic purposes as disclosed herein. In some embodiments, provided herein pharmaceutical compositions comprising, as the active ingredient, a compound of Formula (I) or a pharmaceutically acceptable salt thereof, in combination with one or more pharmaceutically acceptable carriers (excipients). “Acceptable” means that the carrier must be compatible with the active ingredient of the composition (and preferably, capable of stabilizing the active ingredient) and not deleterious to the subject to be treated. Suitable carriers include microcrystalline cellulose, mannitol, glucose, defatted milk powder, polyvinylpyrrolidone, and starch, or a combination thereof.

Some examples of suitable excipients include, without limitation, lactose, dextrose, sucrose, sorbitol, mannitol, starches, gum acacia, calcium phosphate, alginates, tragacanth, gelatin, calcium silicate, microcrystalline cellulose, polyvinylpyrrolidone, cellulose, water, syrup, and methyl cellulose. The pharmaceutical formulations can additionally include, without limitation, lubricating agents such as talc, magnesium stearate, and mineral oil; wetting agents; emulsifying and suspending agents; preserving agents such as methyl-and propylhydroxy-benzoates; sweetening agents; flavoring agents, or combinations thereof. Sec Remington's Pharmaceutical Sciences, 17^{th} ed., Mack Publishing Company, Easton, Pa., 1985, p. 1418 for more information on acceptable pharmaceutical compositions.

Conventional methods, known to those of ordinary skill in the art of medicine, can be used to administer the pharmaceutical composition to the subject, depending upon the type of disease to be treated or the site of the disease. This composition can also be administered via other conventional routes, e.g., administered orally, parenterally, by inhalation spray, topically, rectally, nasally, buccally, vaginally or via an implanted reservoir. The term “parenteral” as used herein includes subcutaneous, intracutaneous, intravenous, intramuscular, intraarticular, intraarterial, intrasynovial, intrasternal, intrathecal, intralesional, and intracranial injection or infusion techniques. Parenteral administration can be in the form of a single bolus dose, or may be, for example, by a continuous perfusion pump. In addition, it can be administered to the subject via injectable depot routes of administration such as using 1-, 3-, or 6-month depot injectable or biodegradable materials and methods.

Injectable compositions may contain various carriers such as vegetable oils, dimethylactamide, dimethyformamide, ethyl lactate, ethyl carbonate, isopropyl myristate, ethanol, and polyols (glycerol, propylene glycol, liquid polyethylene glycol, and the like). For intravenous injection, water soluble antibodies can be administered by the drip method, whereby a pharmaceutical formulation containing the antibody and a physiologically acceptable excipients is infused. Physiologically acceptable excipients may include, for example, 5% dextrose, 0.9% saline, Ringer's solution, or other suitable excipients. Intramuscular preparations, e.g., a sterile formulation of a suitable soluble salt form of the antibody, can be dissolved and administered in a pharmaceutical excipient such as Water-for-Injection, 0.9% saline, or 5% glucose solution.

For oral administration, the composition can take the form of, for example, tablets or capsules, prepared by conventional means with acceptable excipients such as binding agents (for example, pre-gelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulphate). The tablets can be coated by methods well known in the art.

In some embodiments, the above compounds, or a pharmaceutically acceptable salt thereof, provided herein are suitable for parenteral administration. In some embodiments, the above compounds, or a pharmaceutically acceptable salt thereof, are suitable for intravenous administration.

Pharmaceutical compositions and formulations for topical administration may include transdermal patches, ointments, lotions, creams, gels, drops, suppositories, sprays, liquids, and powders. Conventional pharmaceutical carriers, aqueous, powder or oily bases, thickeners and the like may be necessary or desirable.

In making the pharmaceutical compositions provided herein, the active ingredient is typically mixed with an excipient, diluted by an excipient, or enclosed within such a carrier in the form of, for example, a capsule, sachet, paper, or other container. When the excipient serves as a diluent, it can be a solid, semi-solid, or liquid material, which acts as a vehicle, carrier or medium for the active ingredient.

Thus, the pharmaceutical compositions can be in the form of tablets, pills, powders, lozenges, sachets, cachets, elixirs, suspensions, emulsions, solutions, syrups, aerosols (as a solid or in a liquid medium), ointments, soft and hard gelatin capsules, suppositories, sterile injectable solutions, and sterile packaged powders.

### III. Methods of Uses

Any of the granzyme B-targeting therapeutic compound of Formula (I) as disclosed herein may be used in treatment of granzyme B-associated diseases, for example, in cancer therapy.

To practice the methods disclosed herein, an effective amount of a pharmaceutical composition comprising a compound of Formula (I) as disclosed herein, or a pharmaceutically acceptable salt thereof may be administered to a subject in need of the treatment via a suitable route. In some embodiments, such a method may further comprise administering to the subject an effective amount of a granzyme B-targeting agent, for example, any of the compounds of Formula (I) disclosed herein or a pharmaceutically acceptable salt thereof, which may be performed prior to administration of the therapeutic agent. In some embodiments, the subject may have been undergone or is undergoing an immune therapy, e.g., those disclosed herein.

As used herein, the term “subject,” refers to any mammals, for example, mice, rats, other rodents, rabbits, dogs, cats, swine, cattle, sheep, horses, non-human primates, or human. In some embodiments, the subject is a human.

In some embodiments, the subject is a human patient having a cancer. In some instances, the cancer is a solid tumor. Examples include, but are not limited to, brain, breast cancer (e.g., HER^{2+,} ER+/PR+/HER²−, or triple-negative breast cancer), cervical cancer (e.g., squamous cell carcinoma of the cervix), colorectal cancer, lung cancer (e.g., non-small cell lung cancer, or small cell lung cancer), melanoma, bladder cancer, renal cell carcinoma, multiple myeloma, pancreatic cancer, prostate cancer, glioblastoma, hepatocellular carcinoma, urothelial carcinoma, esophageal carcinoma, gastroesophageal carcinoma, gastric cancer, squamous cell carcinoma of the head and neck, epithelial ovarian cancer (EOC), primary peritoneal cancer, fallopian tube carcinoma, Merkel cell cancer, nasopharyngeal cancer, adrenocortical carcinoma, meningioma, neuroblastoma, retinoblastoma, osteosarcoma, rhabdomyosarcoma, Ewing's sarcoma, liposarcoma, fibrosarcoma, leiomyosarcoma, peripheral primitive neuroectodermal tumor, squamous cell carcinoma of the vagina, and squamous cell carcinoma of the vulva. In some examples, the cancer is colon cancer.

In other embodiments, the cancer is a hematological cancer (e.g., leukemia, lymphoma, and the like). Examples include, but are not limited to, Hairy-cell leukemia, Kaposi's sarcoma, follicular lymphoma, chronic myeloid leukemia, cutaneous T-cell lymphoma, peripheral T-cell lymphoma, T-cell prolymphocytic leukemia, Classical Hodgkin's lymphoma, B-cell non-Hodgkin's lymphoma, chronic lymphocytic leukemia, acute myeloid leukemia, myelodysplastic syndrome, primary myelofibrosis, post-essential thrombocytheia myelofibrosis, or post-polycythemia vera myelofibrosis.

As used herein, “an effective amount” refers to the amount of each active agent required to confer therapeutic effect on the subject, either alone or in combination with one or more other active agents. Determination of whether an amount of the antibody achieved the therapeutic effect would be evident to one of skill in the art. Effective amounts vary, as recognized by those skilled in the art, depending on the particular condition being treated, the severity of the condition, the individual patient parameters including age, physical condition, size, gender and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment.

In some examples, a maximum tolerable dose for a checkpoint inhibitor can be used in a method disclosed herein. Alternatively or in addition, a minimum effective dose for any of the therapeutic granzyme B-targeting molecule of Formula (I) disclosed herein may be used in such a method.

Empirical considerations, such as the half-life, generally will contribute to the determination of the dosage. For example, antibodies that are compatible with the human immune system, such as humanized antibodies or fully human antibodies, may be used to prolong half-life of the antibody and to prevent the antibody being attacked by the host's immune system. Frequency of administration may be determined and adjusted over the course of therapy, and is generally, but not necessarily, based on treatment and/or suppression and/or amelioration and/or delay of a target disease/disorder. Alternatively, sustained continuous release formulations of an antibody may be appropriate. Various formulations and devices for achieving sustained release are known in the art.

In some embodiments, the dosage of one of the above compounds, or a pharmaceutically acceptable salt thereof, administered to a subject or individual is about 1 μg to about 2 g, for example, about 1 μg to about 2 g, about 1 μg to about 1000 mg, about 1 μg to about 500 mg, about 1 μg to about 100 mg, about 1 μg to about 50 mg, about 1 μg to about 1 mg, about 1 μg to about 500 μg, about 1 μg to about 100 μg, about 1 μg to about 10 μg, about 10 μg to about 2 g, for example, about 10 μg to about 2 g, about 10 μg to about 1000 mg, about 10 μg to about 500 mg, about 10 μg to about 100 mg, about 10 μg to about 50 mg, about 10 μg to about 1 mg, about 10 μg to about 500 μg, about 10 μg to about 100 μg, about 100 μg to about 2 g, for example, about 100 μg to about 2 g, about 100 μg to about 1000 mg, about 100 μg to about 500 mg, about 100 μg to about 100 mg, about 100 μg to about 50 mg, about 100 μg to about 1 mg, about 100 μg to about 500 μg, about 500 μg to about 2 g, for example, about 500 μg to about 2 g, about 500 μg to about 1000 mg, about 500 μg to about 500 mg, about 500 μg to about 100 mg, about 500 μg to about 50 mg, about 500 μg to about 1 mg, about 1 mg to about 2 g, about 1 mg to about 1000 mg, about 1 mg to about 500 mg, about 1 mg to about 100 mg, about 1 mg to 50 mg, or about 50 mg to about 500 mg.

As used herein, the term “treating” or “treatment” refers to one or more of (1) inhibiting cancer; for example, inhibiting cancer in an individual who is experiencing or displaying the pathology or symptomatology of cancer (i.e., arresting further development of the pathology and/or symptomatology); and (2) ameliorating cancer; for example, ameliorating cancer in an individual who is experiencing or displaying the pathology or symptomatology of cancer (i.e., reversing the pathology and/or symptomatology) such as decreasing the severity of cancer or reducing or alleviating one or more symptoms of cancer.

In some embodiments, the subject for treatment can be identified and/or diagnosed as having the cancer prior to administration of the therapeutic compound of Formula (I). In some instances, a subject having a target cancer can be identified by routine medical examination, e.g., laboratory tests, organ functional tests, CT scans, or ultrasounds. In some embodiments, the subject to be treated by the method described herein may be a human cancer patient who has undergone or is subjecting to an anti-cancer therapy, for example, chemotherapy, radiotherapy, immunotherapy, or surgery.

The human patient subject to any of the cancer therapy disclosed herein (e.g., a theranostic therapy as disclosed herein) may have a prior treatment involving an immune therapeutic agent or is subject to an immune therapy concurrently. An immunotherapeutic agent generally triggers immune effector cells and molecules to target and destroy cells (e.g., cancer cells). The immune effector may be, for example, an antibody specific for a marker on the surface of a cell (e.g., a tumor cell). The antibody alone may serve as an effector of therapy or it may recruit other cells to effect cell killing. Various effector cells include, but are not limited to, cytotoxic T cells and NK cells.

Exemplary immunotherapeutic agents include, but are not limited to, azathioprine, chlorambucil, cyclophosphamide, cyclosporine, daclizumab, infliximab, methotrexate, tacrolimus, immune stimulators (e.g., IL-2, IL-4, IL-12, GM-CSF, tumor necrosis factor; interferons alpha, beta, and gamma; F42K and other cytokine analogs; a chemokine such as MIP-1, MIP-1β, MCP-1, RANTES, IL-8; or a growth factor such FLT3 ligand), an antigenic peptide, polypeptide or protein, or an autologous or allogenic tumor cell composition (see e.g., Ravindranath & Morton, International reviews of immunology, 7.4 (1991): 303-329), hormonal therapy, adrenocorticosteroids, progestins (e.g., hydroxyprogesterone caproate, medroxyprogesterone acetate, and megestrol acetate), estrogens (e.g., diethylstilbestrol and ethinyl estradiol), anti-estrogens (e.g., testosterone propionate and fluoxymesterone), anti-androgens (e.g., flutamide), and gonadotropin-releasing hormone analogs (e.g., leuprolide). Additional immunotherapeutic agents are known in the art, and can be found, for example, in Rosenberg et al, New England Journal of Medicine, 319.25 (1988): 1676-1680; and Rosenberg et al, Annals of surgery, 210.4 (1989): 474).

In some embodiments, the immunetherapeutic agent is an immune checkpoint inhibitor, for example, a PD1 inhibitor (e.g., anti-PD-1 antibodies such as nivolumab, pembrolizumab, or cemiplimab; or anti-PD-L1 antibodies, such as atezolizumab, avelumab, or durvalumab), a CTLA-4 inhibitor (e.g., anti-CTLA-4 antibodies such as ipilimumab), or a LAG-3 inhibitor (e.g., anti-LAG-3 antibody such as relatlimab). In other embodiments, the immunotherapeutic agent can be CAR-T cells, for example, axicabtagene ciloleucel or brexucabtagene autoleucel.

The therapeutic agents provided herein can be effective over a wide dosage range and are generally administered in an effective amount. It will be understood, however, that the amount of the therapeutic agent actually administered will usually be determined by a physician, according to the relevant circumstances, including the condition to be imaged, the chosen route of administration, the actual compound administered, the age, weight, and response of the individual subject, the severity of the subject's symptoms, and the like.

In some embodiments, any of the Granzyme B-target theranostic compounds can be co-used with an anti-cancer therapy, for example, those disclosed herein, e.g., immunotherapy, chemotherapy, etc.

Alternatively or in addition, the Granzyme B-target theranostic compound disclosed herein may be co-used with a Granzyme B-imaging agent. Any imaging compound known in the art that targets granzyme B can be used in the theranostic therapy disclosed herein.

Any of the Granzyme B imaging agents known in the art can be used in the methods disclosed herein. Examples include those disclosed in U.S. Pat. No. 11,559,590, WO2021/252664, PCT/US2022/081098, and PCT/US2022/081125, the relevant disclosures of each of which are incorporated by reference for the subject matter and purpose referenced herein.

In some embodiments, the granzyme B-imaging compound may be a compound of Formula (II), shown below:

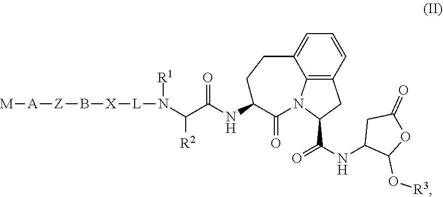

or a stereoisomer, tautomer, or a salt thereof. In Formula (II), M is an imagining agent; A is a chelating moiety chelating the imagining agent; B is aryl, heteroaryl, cycloalkyl, or heterocyclyl (e.g., B is a 6-membered ring); X is —CH₂C(NH)—, —CH₂C(O)—, —CH₂C(S)—, —NHC(NH)—, —NHC(O)—, NHC(S)—, —OC(NH)—, —OC(O)—, or —OC(S)— (e.g., —CH₂C(O)— or —NHC(S)—; Z is —CH₂—, —CH₂C(NH)—, —CH₂C(O)—, —CH₂C(S)—, —NHC(NH)—, —NHC(O)—, —NHC(S)—, —OC(NH)—, —OC(O)—, or —OC(S)—; L is a peptide linker having 1-6 amino acid residues, inclusive; R¹ is H or C₁₋₆ alkyl, optionally wherein R¹ is H or methyl; R² is C₁₋₆ alkyl or C₃₋₆ cycloalkyl; and R³ is C₁₋₆ alkyl.

In some examples, the granzyme B-imaging compound may have the structure of Formula (II-A) shown below:

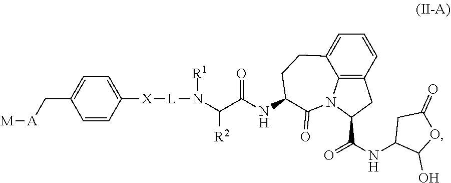

in which M, A, X, L, R^{1,} and R² are as defined above.

In some examples, the granzyme B-imaging compound may have the structure of Formula (II-B) shown below:

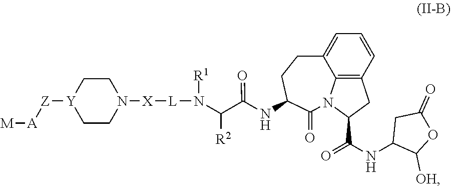

in which M, Z, A, X, L, R¹ and R² are as defined above, and Y is CH or N.

In any of the imaging agents disclosed herein, M can be a metal or a metal linked to a radioisotope suitable for imaging. Suitable metals for use in the present disclosure include those which are useful in imaging Granzyme B, for instance metals which are suitable radioimaging agents, as well as metals which can bind non-metal radioisotopes which are suitable radioimaging agents. An exemplary metal radioisotope is ⁶⁸Ga. An exemplary non-metallic radioisotope is ¹⁸F, which may be conjugated with Al for loading into the Granzyme B binding compounds disclosed herein.

### IV. Kits for Granzyme B-Targeting Theranostic Therapy

Also encompassed by the disclosure are kits (e.g., pharmaceutical packs) for granzyme B-targeting cancer therapy. The kits provided may comprise a container (e.g., a vial, ampule, bottle, syringe, and/or dispenser package, or other suitable container), in which a pharmaceutical composition as disclosed herein may be placed. In some embodiments, provided kits may optionally further include a second container comprising a pharmaceutical excipient for dilution or suspension of a pharmaceutical composition. In some embodiments, the pharmaceutical composition provided in the first container and the second container are combined to form one unit dosage form. In some embodiments, the kit may comprise additional containers comprising one or more additional therapeutic agents as disclosed herein, for example, immunotherapeutic agents. In some embodiments, a kit may comprise a diagnostic imaging compound, or pharmaceutical composition of the same, and a therapeutic compound, or pharmaceutical composition of the same.

In certain embodiments, a kit described herein further includes instructions for using the compounds, agents and compositions included in the kit. A kit described herein may also include information as required by a regulatory agency such as the U.S. Food and Drug Administration (FDA). In certain embodiments, the information included in the kits is prescribing information. In certain embodiments, the kits and instructions provide for imaging Granzyme B and/or treating and/or reducing the risk of cancer in a subject in need thereof. A kit described herein may include one or more additional pharmaceutical agents described herein as a separate composition.

Without further elaboration, it is believed that one skilled in the art can, based on the above description, utilize the present invention to its fullest extent. The following specific embodiments are, therefore, to be construed as merely illustrative, and not limitative of the remainder of the disclosure in any way whatsoever. All publications cited herein are incorporated by reference for the purposes or subject matter referenced herein.

### EXAMPLE 1: SYNTHESIS OF EXEMPLARY COMPOUNDS OF FORMULA (I′)

This Example provides exemplary synthesis methods for making precursor compounds of Formula (I′), which lacks the radioactive moiety relative to corresponding Formula (I) compounds.

### Preparation of Chelating Moiety Coupling Partner

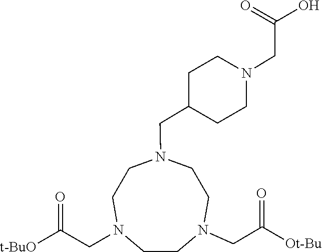 [SMILES: CC(C)(C)OC(=O)CN1CCN(CC(=O)OC(C)(C)C)CCN(CC2CCN(CC(=O)O)CC2)CC1]

2-(4-((4,7-bis(2-(tert-butoxy)-2-oxoethyl)-1,4,7-triazonan-1-yl)methyl)piperidin-1-yl)acetic acid

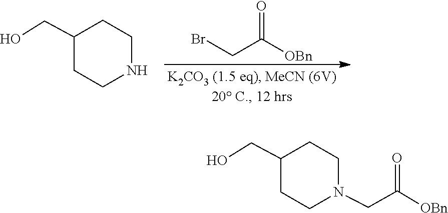 [SMILES: OCC1CCNCC1 ; O=C(CBr)OCc1ccccc1 ; O=C(CN1CCC(CO)CC1)OCc1ccccc1]

Step 1: To a solution of piperidin-4-ylmethanol (90.0 g, 781 mmol) in MeCN (540 mL) was added benzyl 2-bromoacetate (179 g, 781 mmol, 123 mL) and K₂CO₃ (162 g, 1.17 mol). The mixture was stirred at 20° C. for 12 hrs. TLC(petroleum ether:ethyl acetate =0:1, R_{f}=0.20) showed the starting material was consumed completely. The mixture was filtered, and the filtrate was concentrated in vacuum. The residue was purified by column chromatography (SiO₂, petroleum ether:ethyl acetate=50:1 to 0:1). Benzyl 2-(4-(hydroxymethyl)piperidin-1-yl)acetate (92.0 g, 349 mmol) was obtained as yellow oil.

¹H NMR: (400 MHZ, CDCl₃) δ 7.35-7.37 (m, 5H), 5.16 (s, 2H), 3.49 (d, J=6.4 Hz, 2H), 3.26 (s, 2H), 2.95 (d, J=11.2 Hz, 2H), 2.16-2.22 (m, 2H), 1.72 (d, J=13.2 Hz, 2H), 1.59 (s, 1H), 1.49-1.55 (m, 1H), 1.30-1.40 (m, 2H).

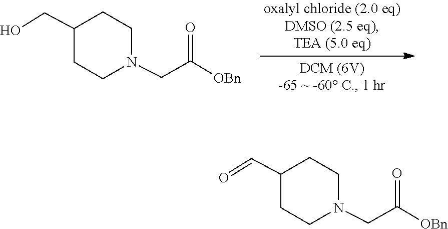 [SMILES: O=C(CN1CCC(CO)CC1)OCc1ccccc1 ; O=CC1CCN(CC(=O)OCc2ccccc2)CC1]

Step 2: To a solution of oxalyl chloride (88.7 g, 699 mmol, 61.2 mL) in DCM (460 mL) was added DMSO (68.2 g, 873 mmol, 68.2 mL) dropwise at −65° C. and the mixture was stirred at −65° C. for 30 mins. A solution of benzyl 2-(4-(hydroxymethyl)piperidin-1-yl)acetate (92.0 g, 349 mmol) in DCM (92.0 mL) was added dropwise below −60° C., followed by TEA (177 g, 1.75 mol, 243 mL). The resulting mixture was stirred at −65° C. for another 30 min. TLC (dichloromethane:methanol=10:1, Rf=0.33) showed the starting material was consumed completely. The reaction mixture was quenched by addition NaHCO₃ (500 mL) at 0° C., and then extracted once with DCM (100 mL). The combined organic layers were washed with once brine (100 mL), dried over anhydrous Na₂SO₄, filtered, and concentrated in vacuum. Benzyl 2-(4-formylpiperidin-1-yl)acetate (135 g, crude) was obtained as yellow oil.

¹H NMR: 400 MHZ, CDCl₃ δ 9.65 (d, J=1.2 Hz, 1H), 7.33-7.38 (m, 5H), 5.17 (s, 2H), 3.29 (s, 2H), 2.86-2.91 (m, 2H), 2.34-2.40 (m, 2H), 2.19-2.28 (m, 1H), 1.90-1.95 (m, 2H), 1.70-1.80 (m, 2H).

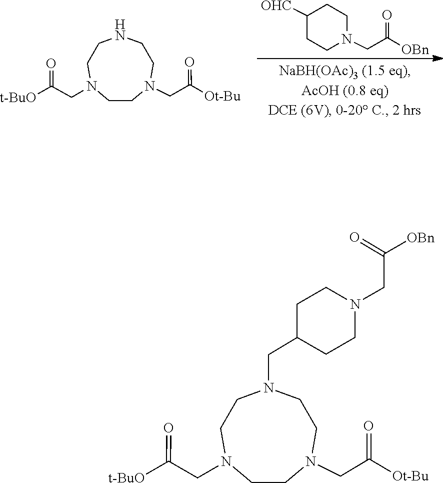 [SMILES: CC(C)(C)OC(=O)CN1CCNCCN(CC(=O)OC(C)(C)C)CC1 ; O=CC1CCN(CC(=O)OCc2ccccc2)CC1 ; CC(C)(C)OC(=O)CN1CCN(CC(=O)OC(C)(C)C)CCN(CC2CCN(CC(=O)OCc3ccccc3)CC2)CC1]

Step 3: A mixture of benzyl 2-(4-formylpiperidin-1-yl)acetate (28.1 g, 107 mmol) in DCE (192 mL) was added di-tert-butyl 2,2′-(1,4,7-triazonane-1,4-diyl)diacetate (32.0 g, 89.5 mmol) and AcOH (4.30 g, 71.6 mmol, 4.10 mL) at 0° C. The mixture was stirred for 1 hr at 0° C. Then NaBH (OAc)₃ (28.5 g, 134 mmol, 1.50 eq) was added in portions. The mixture was stirred at 20° C. for 1 hr. The reaction mixture was quenched by addition NaHCO₃ (200 mL), and then extracted five times with DCM (50.0 mL). The combined organic layers were washed with once brine (50.0 mL), dried over Na₂SO₄, filtered, and concentrated under reduced pressure to give a residue. The crude product from another two reaction, starting from 30.0 g di-tert-butyl 2,2′-(1,4,7-triazonane-1,4-diyl)diacetate and from 32.0 g di-tert-butyl 2,2′-(1,4,7-triazonane-1,4-diyl)diacetate, respectively, were combined for further purification. The combined crude product was triturated with EtOAc (120 mL) for 12 hrs to afford 51.0 g of di-tert-butyl 2,2′-(7-((1-(2-(benzyloxy)-2-oxoethyl)piperidin-4-yl)methyl)-1,4,7-triazonane-1,4-diyl)diacetate as white solid.

ES/MS m/z 603.5 (M+H)⁺.

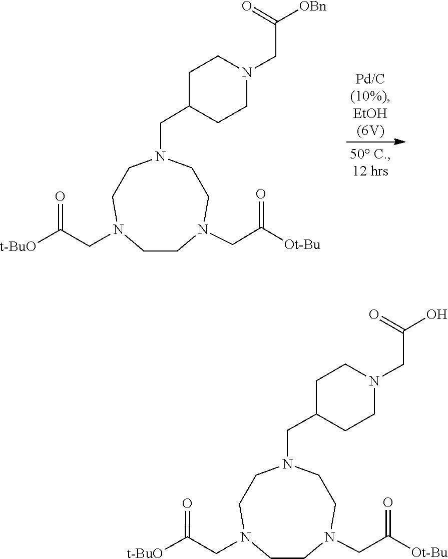 [SMILES: CC(C)(C)OC(=O)CN1CCN(CC(=O)OC(C)(C)C)CCN(CC2CCN(CC(=O)OCc3ccccc3)CC2)CC1 ; CC(C)(C)OC(=O)CN1CCN(CC(=O)OC(C)(C)C)CCN(CC2CCN(CC(=O)O)CC2)CC1]

Step 4: To a solution of di-tert-butyl 2,2′-(7-((1-(2-(benzyloxy)-2-oxoethyl)piperidin-4-yl) methyl)-1,4,7-triazonane-1,4-diyl)diacetate (51.0 g, 84.6 mmol) in EtOH (306 mL) was added Pd/C(5.10 g, 10% purity) under N₂ atmosphere. The suspension was degassed and purged with H2 for 3 times. The mixture was stirred under H₂ (50 Psi) at 50° C. for 4 hrs. The mixture was filtered through celite, and the filtrate was concentrated in vacuum. The crude product was triturated with MTBE (150 mL) for 4 hrs. 2-(4-((4,7-bis(2-(tert-butoxy)-2-oxoethyl)-1,4,7-triazonan-1-yl)methyl)piperidin-1-yl)acetic acid (30.0 g, 56.9 mmol) was obtained as off-white foam.

ES/MS m/z 513.4 (M+H)⁺.

### General Peptide Synthesis Procedure

Peptides were synthesized following standard Fmoc solid-phase peptide synthesis procedures using H-Asp (OtBu)-H resin. Final peptides were deprotected and cleaved from the resin following a two-step procedure: 1) treatment with either trifluoroacetic acid (TFA) at room temperature for 2 h or TFA/dichloromethane (DCM) at room temperature overnight, then concentrated; 2) treatment with 0.1% TFA in acetonitrile/water (60:40) at 60° C. for 1 h (https://www.emdmillipore.com/US/en/product/H-AspOtBu-H-NovaSyn-TG-resin,MDA_CHEM-856072 #documentation). Crude peptides were either concentrated or lyophilized, then subjected to preparative HPLC purification (0.1% formic acid or 0.1% TFA in water/acetonitrile mobile phase). Product-containing fractions were collected and lyophilized to afford peptides as white, fluffy solids.

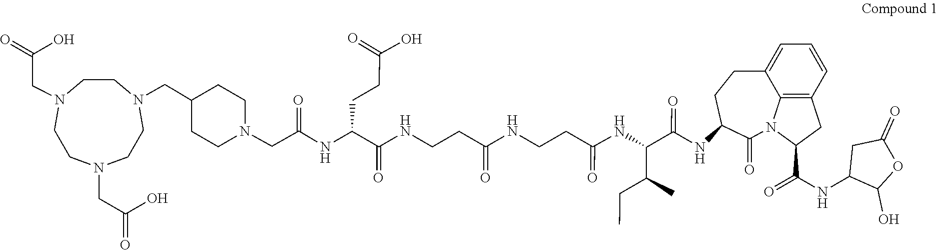 [SMILES: CC[C@H](C)[C@H](NC(=O)CCNC(=O)CCNC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (36 mg) was prepared.

ES/MS m/z 1112.6 (M+H)⁺.

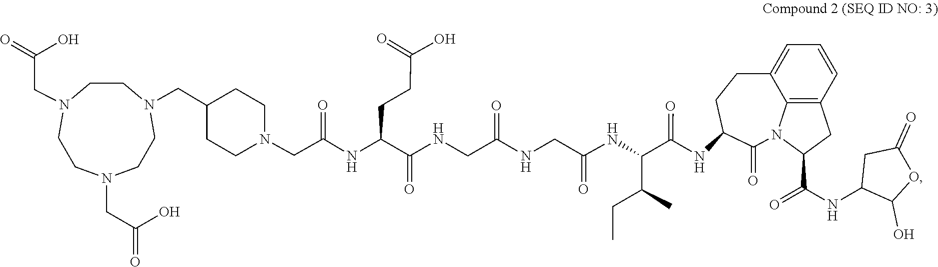 [SMILES: CC[C@H](C)[C@H](NC(=O)CNC(=O)CNC(=O)[C@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperidin-4-yl)methyl)-1,4,7-triazonane-1,4-diyl)diacetic acid (100 mg) was obtained.

ES/MS m/z 1084.5 (M+H)⁺.

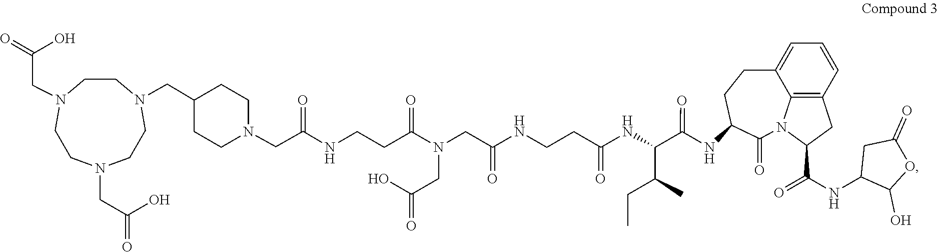 [SMILES: CC[C@H](C)[C@H](NC(=O)CCNC(=O)CN(CC(=O)O)C(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((15S,16S)-7-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (49 mg) was prepared.

ES/MS m/z 1098.4 (M+H)⁺.

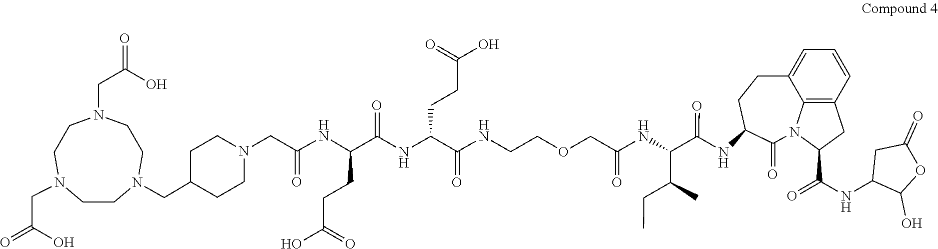 [SMILES: CC[C@H](C)[C@H](NC(=O)COCCNC(=O)[C@@H](CCC(=O)O)NC(=O)[C@@H](CCC(=O)O)NC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperidin-4-yl)methyl)-1,4,7-triazonane-1,4-diyl)diacetic acid (80 mg) was obtained.

ES/MS m/z 1200.6 (M+H)⁺.

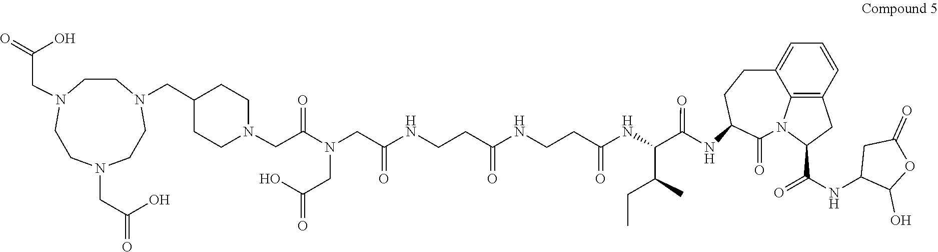 [SMILES: CC[C@H](C)[C@H](NC(=O)CCNC(=O)CCNC(=O)CN(CC(=O)O)C(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperidin-4-yl)methyl)-1,4,7-triazonane-1,4-diyl)diacetic acid (28 mg) was obtained.

ES/MS m/z 1098.6 (M+H)⁺.

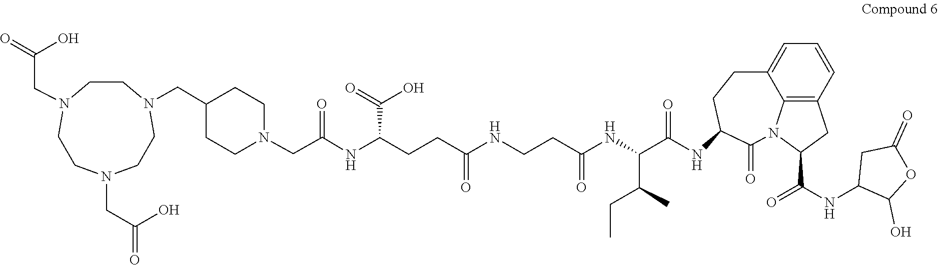 [SMILES: CC[C@H](C)[C@H](NC(=O)CCNC(=O)CC[C@H](NC(=O)CN1CCC(CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-((1-(2-(((1S)-1-carboxy-4-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-4-oxobutyl)amino)-2-oxoethyl)piperidin-4-yl)methyl)-1,4,7-triazonane-1,4-diyl)diacetic acid (32 mg) was obtained.

ES/MS m/z 1041.6 (M+H)⁺.

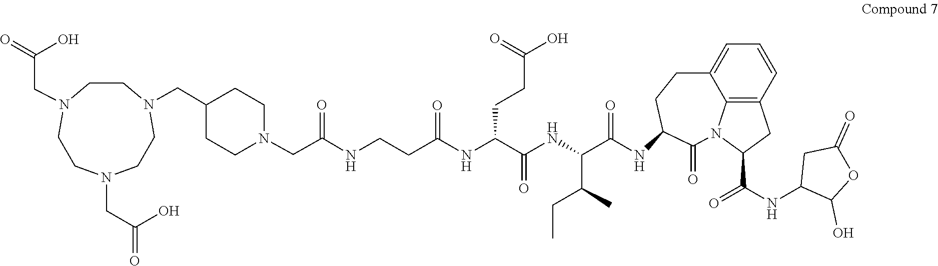 [SMILES: CC[C@H](C)[C@H](NC(=O)[C@@H](CCC(=O)O)NC(=O)CCNC(=O)CN1CCC(CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-((1-((8R,15S,16S)-8-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi] indol-3-yl)carbamoyl)-16-methyl-2,6,9,13-tetraoxo-3,7,10,14-tetraazaoctadecyl)piperidin-4-yl)methyl)-1,4,7-triazonane-1,4-diyl)diacetic acid (24.6 mg) was obtained.

ES/MS m/z 1112.5 (M+H)⁺.

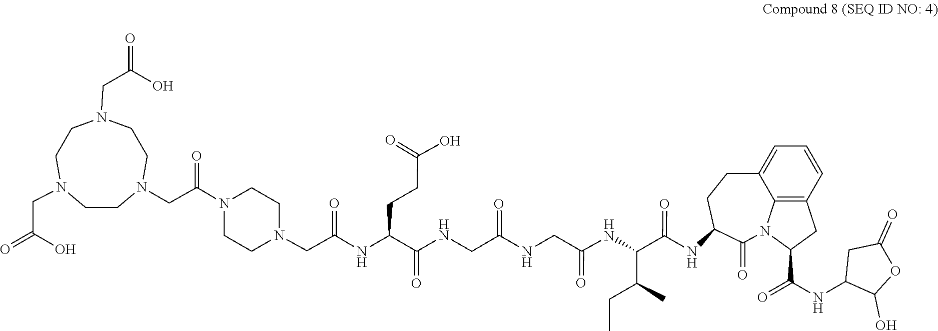 [SMILES: CC[C@H](C)[C@H](NC(=O)CNC(=O)CNC(=O)[C@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4S,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,8,11-tetraoxo-3,6,9,12-tetraazahexadecyl)piperazin-1-yl)-2-oxoethyl)-1,4,7-triazonane-1,4-diyl)diacetic acid (100 mg) was obtained.

ES/MS m/z 1113.5 (M+H)⁺.

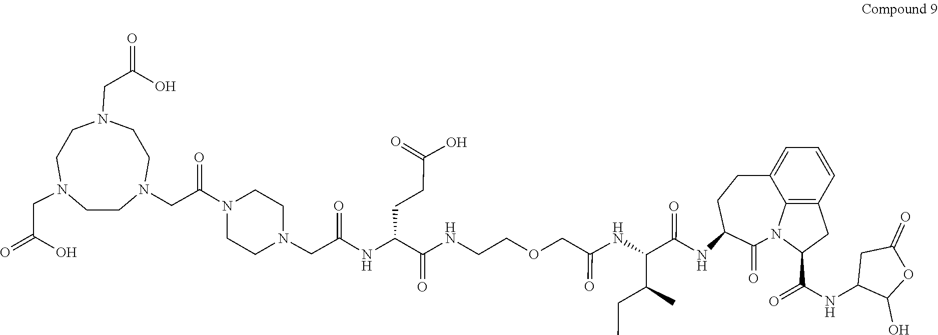 [SMILES: CC[C@H](C)[C@H](NC(=O)COCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,13S,14S)-4-(2-carboxyethyl)-13-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-14-methyl-2,5,11-trioxo-9-oxa-3,6,12-triazahexadecyl)piperazin-1-yl)-2-oxoethyl)-1,4,7-triazonane-1,4-diyl)diacetic acid (78 mg) was obtained.

ES/MS m/z 1100.5 (M+H)⁺.

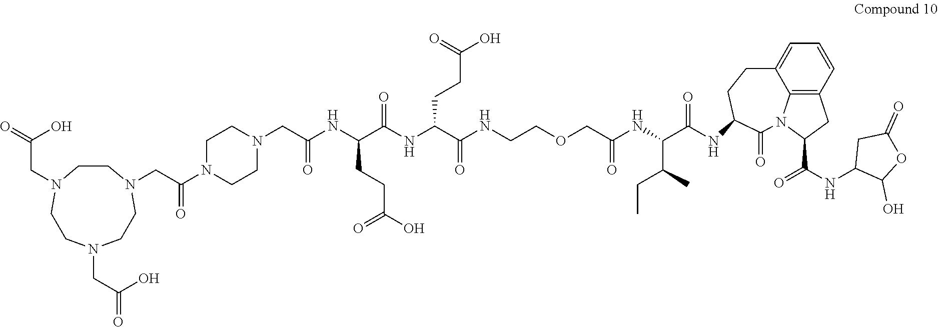 [SMILES: CC[C@H](C)[C@H](NC(=O)COCCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,7R,16S,17S)-4,7-bis(2-carboxyethyl)-16-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-17-methyl-2,5,8,14-tetraoxo-12-oxa-3,6,9,15-tetraazanonadecyl)piperazin-1-yl)-2-oxoethyl)-1,4,7-triazonane-1,4-diyl) diacetic acid (22 mg) was obtained.

ES/MS m/z 1229.7 (M+H)⁺.

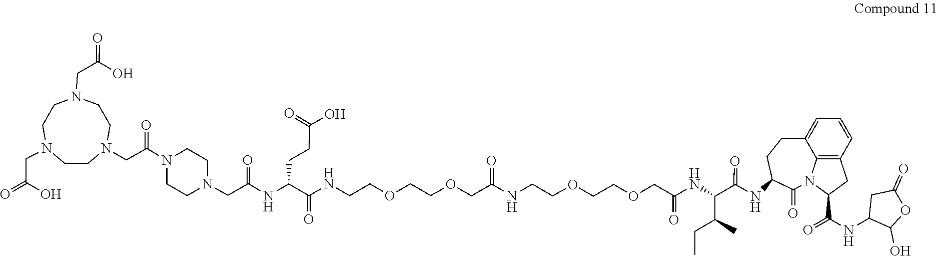 [SMILES: CC[C@H](C)[C@H](NC(=O)COCCOCCNC(=O)COCCOCCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,25S,26S)-4-(2-carboxyethyl)-25-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-26-methyl-2,5,14,23-tetraoxo-9,12,18,21-tetraoxa-3,6,15,24-tetraazaoctacosyl)piperazin-1-yl)-2-oxoethyl)-1,4,7-triazonane-1,4-diyl)diacetic acid (46 mg) was obtained.

ES/MS m/z 1289.6 (M+H)⁺

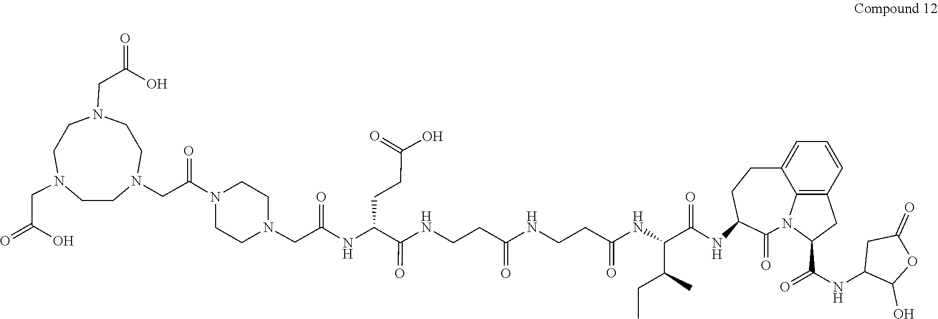 [SMILES: CC[C@H](C)[C@H](NC(=O)CCNC(=O)CCNC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (30 mg) was obtained.

ES/MS m/z 1141.6 (M+H)⁺.

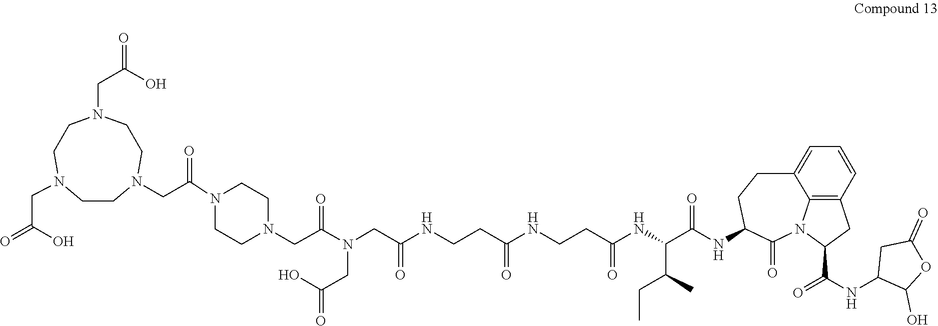 [SMILES: CC[C@H](C)[C@H](NC(=O)CCNC(=O)CCNC(=O)CN(CC(=O)O)C(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((15S,16S)-3-(carboxymethyl)-15-(((3S,6S)-6-((2-hydroxy-5-oxotetrahydrofuran-3-yl)carbamoyl)-4-oxo-1,2,3,4,6,7-hexahydroazepino[3,2,1-hi] indol-3-yl)carbamoyl)-16-methyl-2,5,9,13-tetraoxo-3,6,10,14-tetraazaoctadecyl)piperazin-1-yl)-2-oxoethyl)-1,4,7-triazonane-1,4-diyl)diacetic acid (29 mg) was obtained.

ES/MS m/z 1127.6 (M+H)⁺.

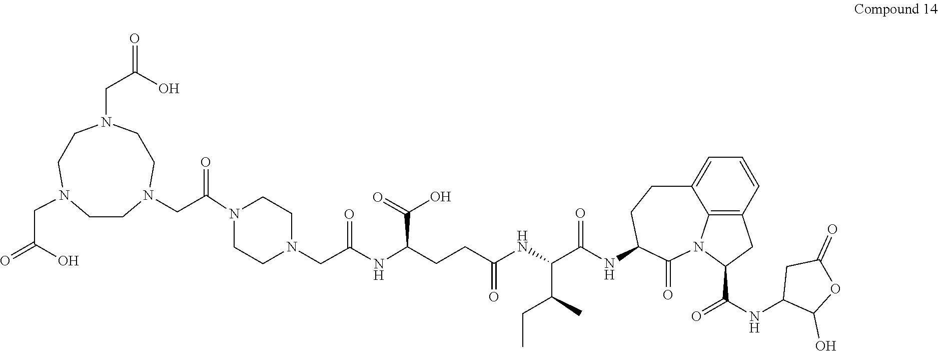 [SMILES: CC[C@H](C)[C@H](NC(=O)CC[C@@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-(2-(4-(2-(((1R)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (32 mg) was obtained.

ES/MS m/z 999.5 (M+H)⁺.

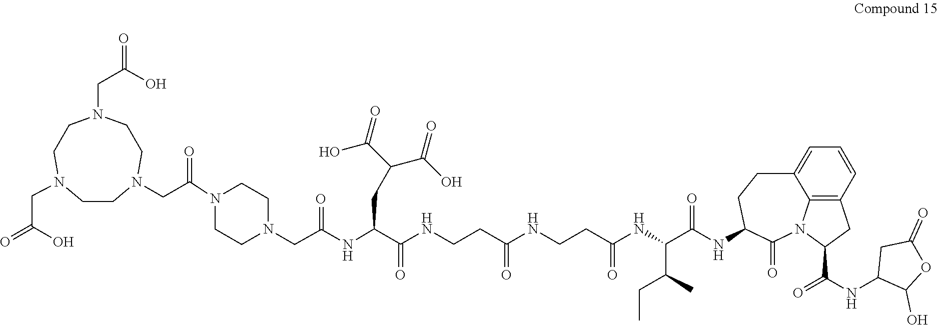 [SMILES: CC[C@H](C)[C@H](NC(=O)CCNC(=O)CCNC(=O)[C@H](CC(C(=O)O)C(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2-((2S)-2-(2-(4-(2-(4,7-bis (carboxymethyl)-1,4,7-triazonan-1-yl)acetyl)piperazin-1-yl)acetamido)-3-((3-((3-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)amino)-3-methyl-1-oxopentan-2-yl)amino)-3-oxopropyl)amino)-3-oxopropyl)amino)-3-oxopropyl)malonic acid (20 mg) was obtained.

ES/MS m/z 1185.6 (M+H)⁺.

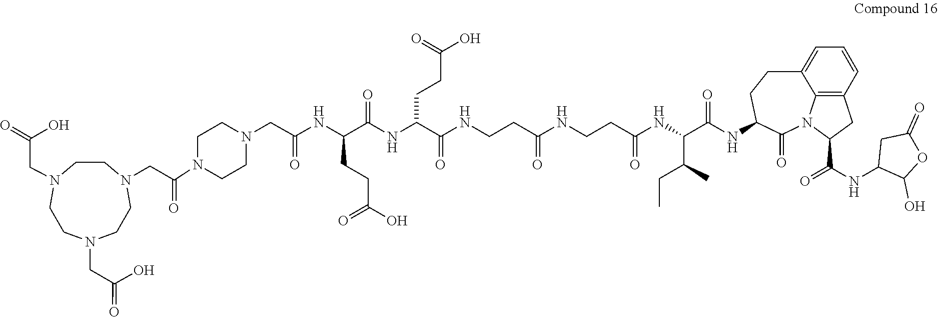 [SMILES: CC[C@H](C)[C@H](NC(=O)CCNC(=O)CCNC(=O)[C@@H](CCC(=O)O)NC(=O)[C@@H](CCC(=O)O)NC(=O)CN1CCN(C(=O)CN2CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′-(7-(2-(4-((4R,7R,18S,19S)-4,7-bis (2-carboxyethyl)-18-(((3S,6S)-6-((2-hydroxy-5-oxotetrahydrofuran-3-yl)carbamoyl)-4-oxo-1,2,3,4,6,7-hexahydroazepino[3,2,1-hi]indol-3-yl)carbamoyl)-19-methyl-2,5,8,12,16-pentaoxo-3,6,9,13,17-pentaazahenicosyl)piperazin-1-yl)-2-oxoethyl)-1,4,7-triazonane-1,4-diyl) diacetic acid (30 mg) was obtained.

ES/MS m/z 1270.6 (M+H)⁺.

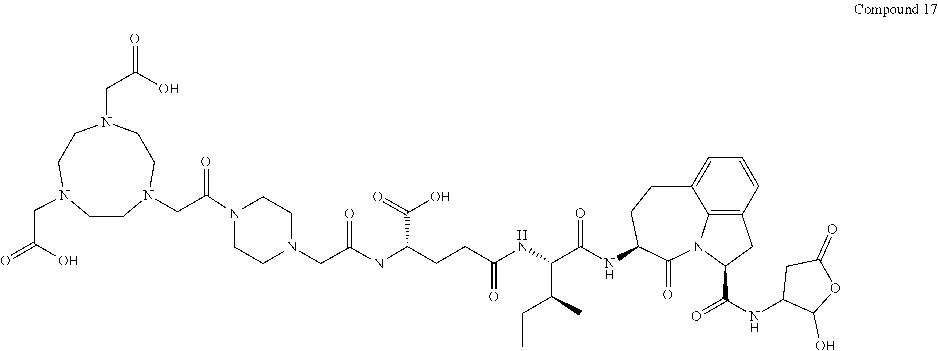 [SMILES: CC[C@H](C)[C@H](NC(=O)CC[C@H](NC(=O)CN1CCN(C(=O)CN2CCN(CC(=O)O)CCN(CC(=O)O)CC2)CC1)C(=O)O)C(=O)N[C@H]1CCc2cccc3c2N(C1=O)[C@H](C(=O)NC1CC(=O)OC1O)C3]

Following the general peptide synthesis procedure, 2,2′-(7-(2-(4-(2-(((1S)-1-carboxy-4-(((2S,3S)-1-(((3S,6S)-6-((2-hydroxy-5-oxotetrahydrofuran-3-yl)carbamoyl)-4-oxo-1,2,3,4,6,7-hexahydroazepino[3,2,1-hi] indol-3-yl)amino)-3-methyl-1-oxopentan-2-yl)amino)-4-oxobutyl)amino)-2-oxoethyl)piperazin-1-yl)-2-oxoethyl)-1,4,7-triazonane-1,4-diyl)diacetic acid (30 mg) was obtained.

ES/MS m/z 999.5 (M+H)⁺.

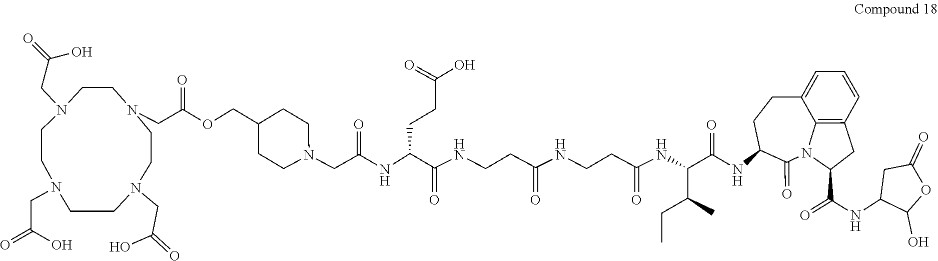 [SMILES: CC[C@H](C)[C@H](NC(=O)CCNC(=O)CCNC(=O)[C@@H](CCC(=O)O)NC(=O)CN1CCC(COC(=O)CN2CCN(CC(=O)O)CCN(CC(=O)O)CCN(CC(=O)O)CC2)CC1)C(=O)N[C@H]1CCc2cccc3c2N(C1=O)[C@H](C(=O)NC1CC(=O)OC1O)C3]

Following the general peptide synthesis procedure, 2,2′,2″-(10-(2-((1-((4R,15S,16S)-4-(2-carboxyethyl)-15-(((3S,6S)-6-((2-hydroxy-5-oxotetrahydrofuran-3-yl)carbamoyl)-4-oxo-1,2,3,4,6,7-hexahydroazepino[3,2,1-hi]-3-yl)carbamoyl)-16-methyl-2,5,9,13-tetraoxo-3,6,10,14-tetraazaoctadecyl)piperidin-4-yl)methoxy)-2-oxoethyl)-1,4,7,10-tetraazacyclododecane-1,4,7-triyl)triacetic acid (30 mg) was obtained.

ES/MS m/z 1270.6 (M+H)⁺.

### EXAMPLE 2: SYNTHESIS OF EXEMPLARY COMPOUNDS OF FORMULA (I)

### General Procedure for Therapeutic Isotope Complexation

To a reaction vial containing peptide precursor and a stir bar, is added equal equivalent (1.5-3.0 equiv relative to peptide) of a suitable amount of a therapeutic radioisotope (e.g., ⁹⁰Y) in a solvent and stirred. This results in a therapeutic isotope complexation.

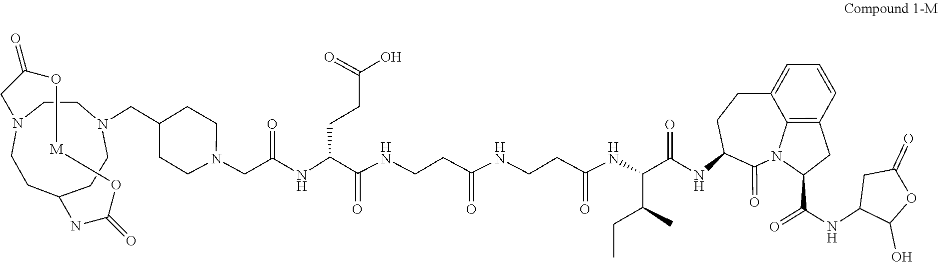

Following the general procedure for the therapeutic isotope complexation, Compound 1-M is synthesized.

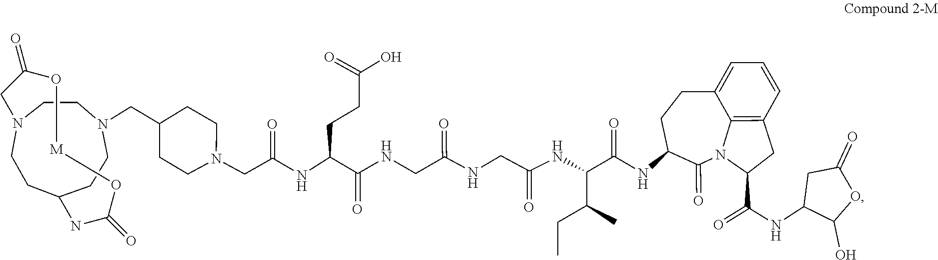

Following the general procedure for the therapeutic isotope complexation, Compound 2-M is synthesized.

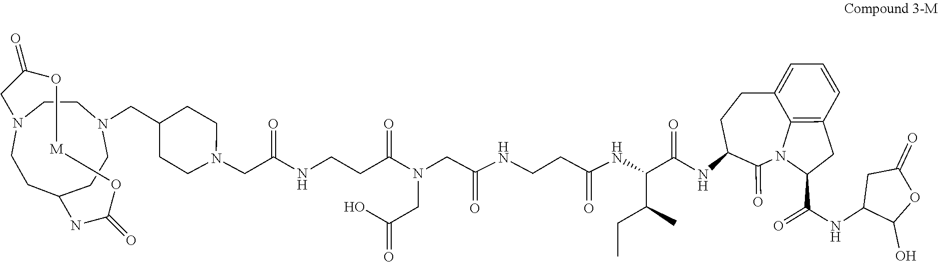

Following the general procedure for the therapeutic isotope complexation, Compound 3-M is synthesized.

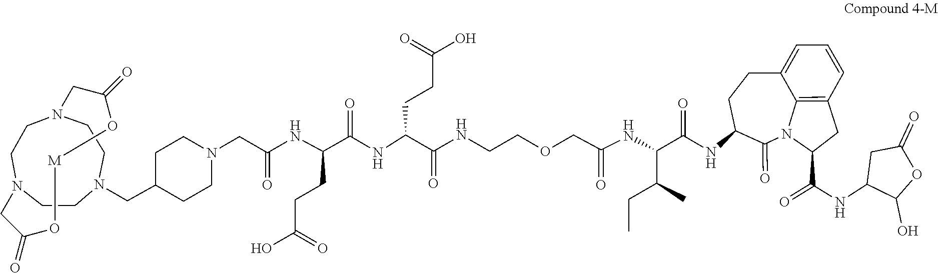

Following the general procedure for the therapeutic isotope complexation, Compound 4-M is synthesized.

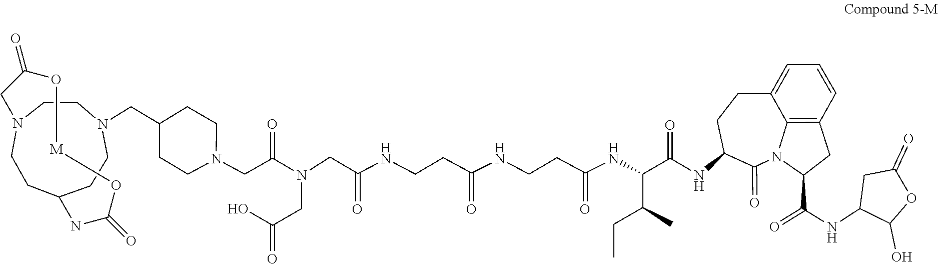

Following the general procedure for the therapeutic isotope complexation, Compound 5-M is synthesized.

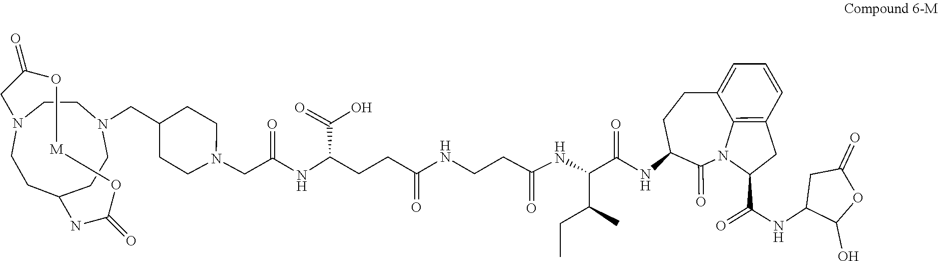

Following the general procedure for the therapeutic isotope complexation, Compound 6-M is synthesized.

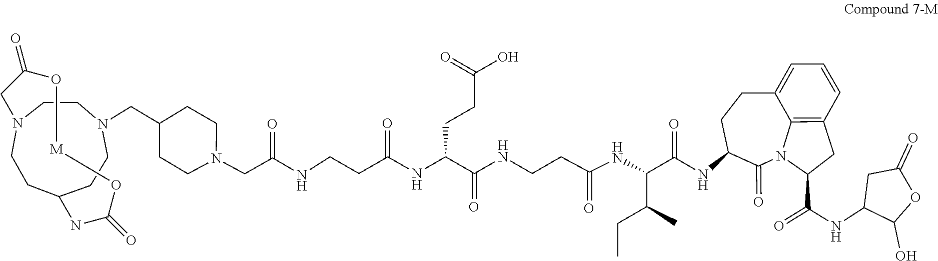

Following the general procedure for the therapeutic isotope complexation, Compound 7-M is synthesized.

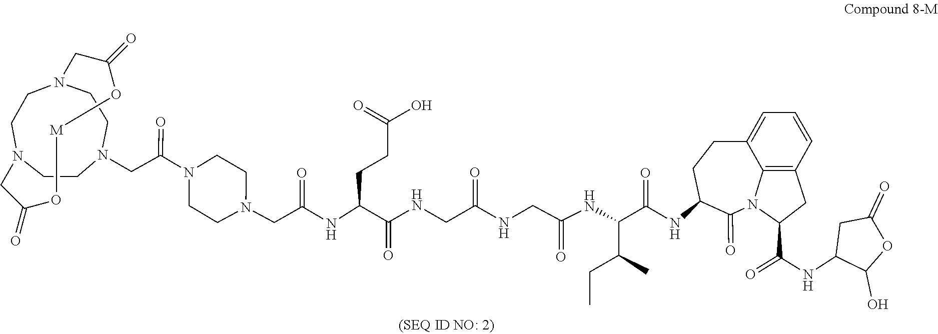

Following the general procedure for the therapeutic isotope complexation, Compound 8-M is synthesized.

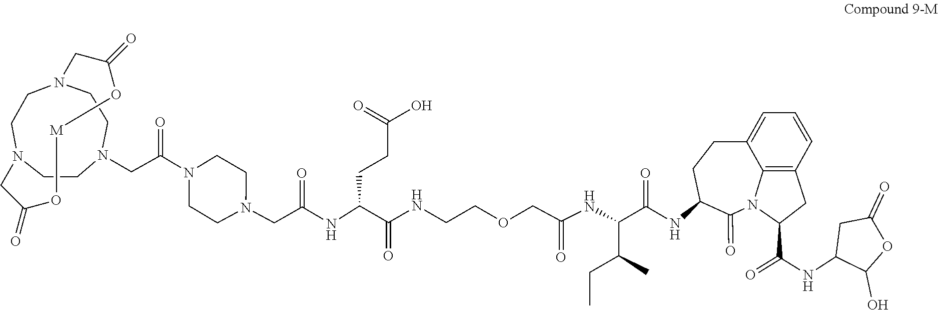

Following the general procedure for the therapeutic isotope complexation, Compound 9-M is synthesized.

[symbol]

### Compound 10-M

Following the general procedure for the therapeutic isotope complexation, Compound 10-M is synthesized.

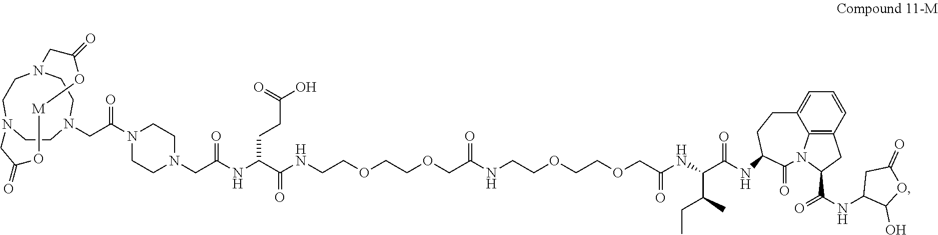

Following the general procedure for the therapeutic isotope complexation, Compound 11-M is synthesized.

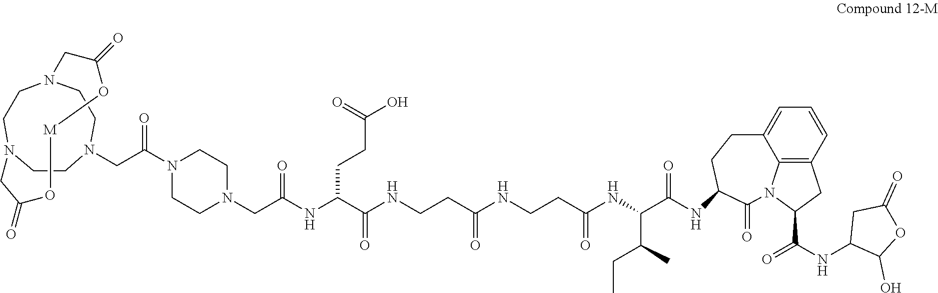

Following the general procedure for the therapeutic isotope complexation, Compound 12-M is synthesized.

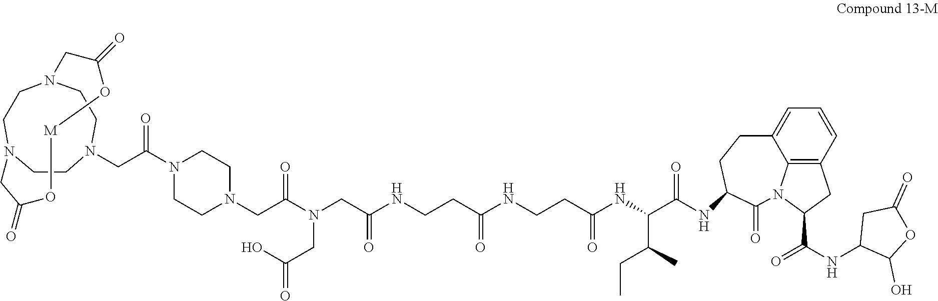

Following the general procedure for the therapeutic isotope complexation, Compound 13-M is synthesized.

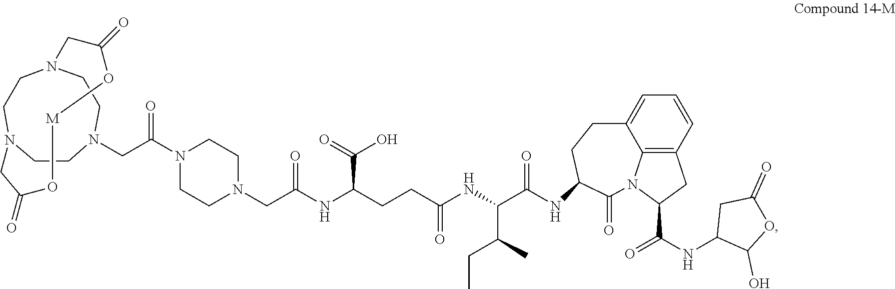

Following the general procedure for the therapeutic isotope complexation, Compound 14-M is synthesized.

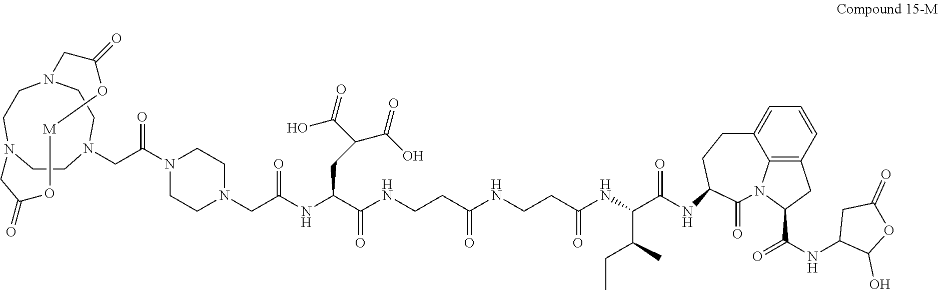

Following the general procedure for the therapeutic isotope complexation, Compound 15-M is synthesized.

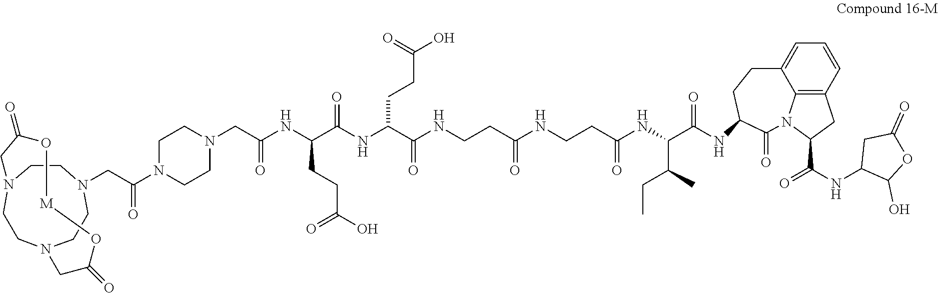

Following the general procedure for the therapeutic isotope complexation, Compound 16-M is synthesized.

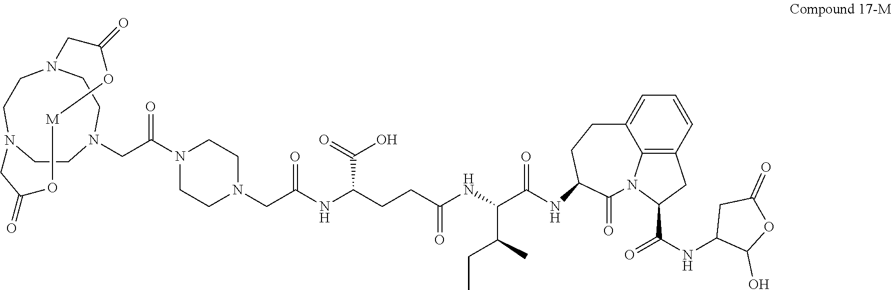

Following the general procedure for the therapeutic isotope complexation, Compound 17-M is synthesized.

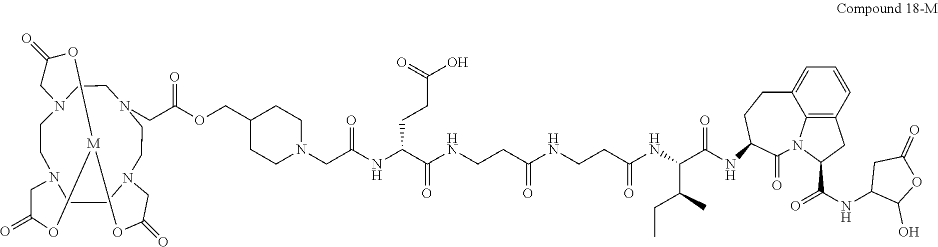

Following the general procedure for the therapeutic isotope complexation, Compound 18-M is synthesized.

### EXAMPLE 3: RADIOSYNTHESIS OF ⁹⁰Y-CONJUGATED COMPOUNDS

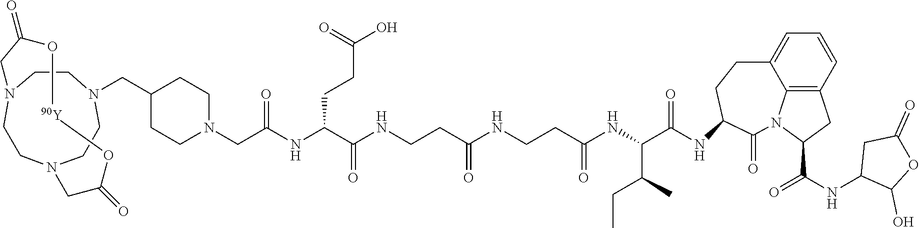

Typical Compound 1-⁹⁰Y RCY synthesized on an ORA Neptis Perform radiosynthesizer ranges from 14-20% (n=7) using 29-55 GBq starting activity. Synthesis time is in 68±5 minutes with product radiochemical purity >93% and specific activity ranging from 167-1320 GBq/μmol (3.9-30.9 mCi/μg).

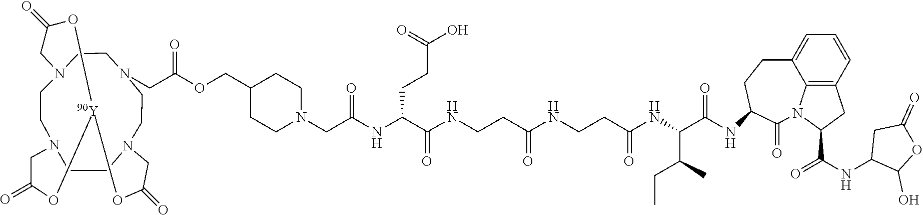

Compound 18-⁹⁰Y is synthesized following the same method as described above.

### EXAMPLE 4: COMPETITIVE BINDING ASSAY OF HUMAN GRANZYME B

Purpose: To perform competitive binding assays with human granzyme B using Compound 1-⁹⁰Y to determine IC₅₀ values of potential granzyme B ligands

Initial Plate Preparation (including total binding and non-specific binding):
1. 78 μL of reaction buffer, as described above, is added to each well in a 96 well plate.
2. For wells used to examine total binding (defined as the binding of the radioligand in the absence of competitors), 2 μL of DMSO is added to give 1% final DMSO concentration for all wells.
3. For wells used to examine non-specific binding (defined as the binding of the radioligand in the presence of 10 μM 21), 2 μL of 1 mM Compound 1 is added to give a final concentration of 10 μM.

Enzyme Preparation:
1. Granzyme B (GZB, human lymphocyte) is diluted to 0.5 μg/mL using reaction buffer as described above.
2. 100 μL of 0.5 μg/mL diluted human GzB is added to each well, giving 50 ng per well and a final concentration of 0.25 μg/mL.

Compound Preparation:
1. DMSO or water is added to the respective compound vial to make 1 mM compound stock solution.
2. 1 mM compound stock solution is serially diluted by ½ log (3.16 fold) by adding 18.5 μL of compound stock to 40 μL of DMSO, mixing by pipetting up and down, and transferring 18.5 μL of the mixture to 40 μL of DMSO. This process is repeated to create 10 dilution points for test compounds.
3. For wells used to examine ligand binding competition, 2 μL of each dilution is dispensed into the 96 well plates prepared as described above, giving a further dilution of 1/100.

Radioligand Preparation:
1. 200 nM of Compound 1-⁹⁰Y (10× radioligand stock) is prepared in reaction buffer, aiming for approximately 2 million CPM per 20 μL of solution (input).
2. 20 μL of radioligand stock is dispensed into each well to give a final concentration per well of 20 nM.

Incubation Conditions, Post-incubation Sample Processing and Data Analysis:
1. All 96 wells should have a final volume of 200 μL, and the assay plates are incubated at 37° C. for 90 minutes.
2. All samples from the assay plates are transferred to MultiScreenHTs FB plates that have been presoaked in PBS buffer, pH 7.4, and filtered using a vacuum manifold. An additional 150 μL of PBS buffer is added to each well in the assay plates and combined to ensure transfer of any remaining samples.
3. MultiScreenHTS FB plates are washed 3× with 150 μL of PBS buffer.
4. All filters are separated and transferred to individual tubes using Multiscreen punch tips and the Millipore multiple punch apparatus.
5. The sample set is analyzed using the Wizard 2480 automatic gamma-counter [Perkin Elmer]. Values are reported as decay-corrected counts per minute (CPM).
6. Resulting CPM values are normalized and converted to % inhibition by the following equation:

$$\% inhibition = \frac{1 - ( sample C P M - N S B C P M )}{T B C P M - N S B C P M}$$

*the total bound fraction is typically less than 10% of the added radioligand under such assay conditions.

Resulting % inhibition values were plotted using the software GraphPad Prism 8.4.3 using the One site—Fit logIC₅₀ equation to determine the IC₅₀ value for each ligand.

$$Y = A + ( B - A ) / ( 1 + 1 {0}^{( x - LogIC 50 )} )$$

Y=% Inhibition
X=Log concentration of the cold competing ligand (M)
A=minimum Y (0%)
B=maximum Y (100%)
LogIC₅₀=Log concentration of cold competing ligand (M) at half-way between minimum and maximum Y

### Other Embodiments

All of the features disclosed in this specification may be combined in any combination. Each feature disclosed in this specification may be replaced by an alternative feature serving the same, equivalent, or similar purpose. Thus, unless expressly stated otherwise, each feature disclosed is only an example of a generic series of equivalent or similar features.

Further, from the above description, one skilled in the art can easily ascertain the essential characteristics of the present disclosure, and without departing from the spirit and scope thereof, can make various changes and modifications of the disclosure to adapt it to various usages and conditions. Thus, other embodiments are also within the claims.

### EQUIVALENTS

While several inventive embodiments have been described and illustrated herein, those of ordinary skill in the art will readily envision a variety of other means and/or structures for performing the function and/or obtaining the results and/or one or more of the advantages described herein, and each of such variations and/or modifications is deemed to be within the scope of the inventive embodiments described herein. More generally, those skilled in the art will readily appreciate that all parameters, dimensions, materials, and configurations described herein are meant to be exemplary and that the actual parameters, dimensions, materials, and/or configurations will depend upon the specific application or applications for which the inventive teachings is/are used. Those skilled in the art will recognize, or be able to ascertain using no more than routine experimentation, many equivalents to the specific inventive embodiments described herein. It is, therefore, to be understood that the foregoing embodiments are presented by way of example only and that, within the scope of the appended claims and equivalents thereto, inventive embodiments may be practiced otherwise than as specifically described and claimed. Inventive embodiments of the present disclosure are directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the inventive scope of the present disclosure.

All definitions, as defined and used herein, should be understood to control over dictionary definitions, definitions in documents incorporated by reference, and/or ordinary meanings of the defined terms.

All references, patents and patent applications disclosed herein are incorporated by reference with respect to the subject matter for which each is cited, which in some cases may encompass the entirety of the document.

The indefinite articles “a” and “an,” as used herein in the specification and in the claims, unless clearly indicated to the contrary, should be understood to mean “at least one.”

The phrase “and/or,” as used herein in the specification and in the claims, should be understood to mean “either or both” of the elements so conjoined, i.e., elements that are conjunctively present in some cases and disjunctively present in other cases. Multiple elements listed with “and/or” should be construed in the same fashion, i.e., “one or more” of the elements so conjoined. Other elements may optionally be present other than the elements specifically identified by the “and/or” clause, whether related or unrelated to those elements specifically identified. Thus, as a non-limiting example, a reference to “A and/or B”, when used in conjunction with open-ended language such as “comprising” can refer, in one embodiment, to A only (optionally including elements other than B); in another embodiment, to B only (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.” “Consisting essentially of,” when used in the claims, shall have its ordinary meaning as used in the field of patent law.

As used herein in the specification and in the claims, the phrase “at least one,” in reference to a list of one or more elements, should be understood to mean at least one element selected from any one or more of the elements in the list of elements, but not necessarily including at least one of each and every element specifically listed within the list of elements and not excluding any combinations of elements in the list of elements. This definition also allows that elements may optionally be present other than the elements specifically identified within the list of elements to which the phrase “at least one” refers, whether related or unrelated to those elements specifically identified. Thus, as a non-limiting example, “at least one of A and B” (or, equivalently, “at least one of A or B,” or, equivalently “at least one of A and/or B”) can refer, in one embodiment, to at least one, optionally including more than one, A, with no B present (and optionally including elements other than B); in another embodiment, to at least one, optionally including more than one, B, with no A present (and optionally including elements other than A); in yet another embodiment, to at least one, optionally including more than one, A, and at least one, optionally including more than one, B (and optionally including other elements); etc.

It should also be understood that, unless clearly indicated to the contrary, in any methods claimed herein that include more than one step or act, the order of the steps or acts of the method is not necessarily limited to the order in which the steps or acts of the method are recited.

1. A compound of formula (I): 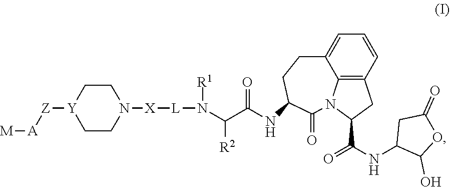
or a stereoisomer, tautomer, or a salt thereof,
wherein:
M is a radioactive moiety;
A is a chelating moiety chelating the radioactive moiety;
X is selected from the group consisting of —CH₂C(NH)—, —CH₂C(O)—, —CH₂C(S)—, —NHC(NH)—, —NHC(O)—, —NHC(S)—, —OC(NH)—, —OC(O)—, and —OC(S)—, optionally wherein X is —CH₂C(O)— or —NHC(S)—;
Y is CH or N;
Z is —CH₂—, —CH₂C(NH)—, —CH₂C(O)—, —CH₂C(S)—, —NHC(NH)—, —NHC(O)—, —NHC(S)—, —O(NH)—, —O—C(O)—, or —OC(S)—; optionally wherein Z is —CH₂— or —CH₂C(O)—;
L is a peptide linker having 1-6 amino acid residues, inclusive;
R¹ is H or C₁₋₆ alkyl, optionally wherein R¹ is H or methyl; and
R² is C₁₋₆ alkyl or C₃₋₆ cycloalkyl.

2. The compound of claim 1, wherein the compound is of Formula (Ia):

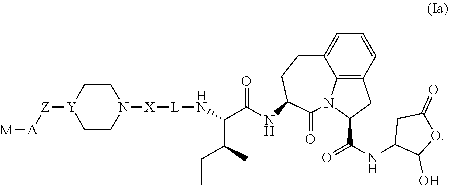

3. The compound of claim 1, wherein X is —CH₂C(O)—.

4. The compound of claim 3, wherein the compound is of Formula (Ib):

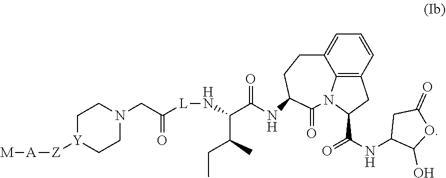

5. The compound of claim 1, wherein L has 1-5 amino acid residues, inclusive; optionally wherein L has one or more non-naturally occurring amino acid residues.

6. The compound of claim 1, wherein L has an amino acid sequence selected from group consisting of:
Glu-Gly-Gly,
Glu-βAla-βAla,
γGlu,
_{D}γGlu,
γGlu-βAla,
_{D}Glu-βAla-βAla,
_{D}Glu-AEA,
_{D}Glu-AEEA-AEEA,
_{D}Glu-_{D}Glu-AEA,
_{D}Glu-_{D}Glu-βAla-βAla,
βAla-_{D}Glu-βAla,
Diacid-βAla-βAla,
N-acid-βAla-βAla, and
βAla-N-acid-βAla.

7. The compound of claim 1, wherein the chelating moiety A is 1,4,7-triazacyclononane-N,N′,N″-triacetic acid (NOTA), 1,4,7-triazacyclononane-4,7-diyl diacetic acid (NODA), 1,4,7,10-tetraazacyclododecane-1,4,7,10-tetraacetic acid (DOTA), 1,4,7,10-Tetraazacyclododecane-1,4,7-triacetic acid (DO3A), 1,4,7,10-Tetraazacyclododecane-1,4,7-triacetic acid (DO3A), Restrained Complexing Agent (RESCA), or MACROPA.

8. The compound of claim 1, wherein the radioactive moiety of M is a therapeutic radioisotope.

9. The compound of claim 8, wherein the therapeutic radioisotope is ⁶⁷Cu, ⁹⁰Y, ¹⁷⁷Lu, ²²⁵Ac, ⁴⁷Sc, ¹³¹I, ¹⁵³Sm, ¹⁶¹Tb, ²¹¹At, ²¹²Pb, ²¹²Bi, ²²³Ra, or ²²⁷Th, optionally wherein the therapeutic radioisotope is ⁹⁰Y.

10. The compound of claim 1, wherein the chelating moiety is NOTA or DOTA, and the therapeutic radioisotope is ⁹⁰Y, ¹⁷⁷Lu, or ²²⁵Ac.

11. The compound of claim 1, wherein the chelating moiety is NODA, and the therapeutic radioisotope is ⁴⁷Sc or ⁶⁷Cu.

12. The compound of claim 1, wherein the compound has one of the following structures: 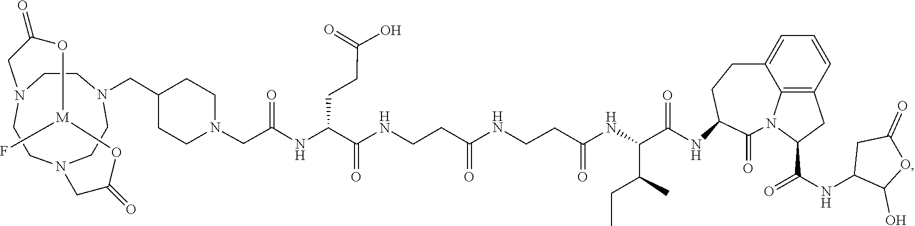
wherein
M is ¹⁷⁷Lu, ⁹⁰Y, ²²⁵Ac, or ²¹³Bi, or 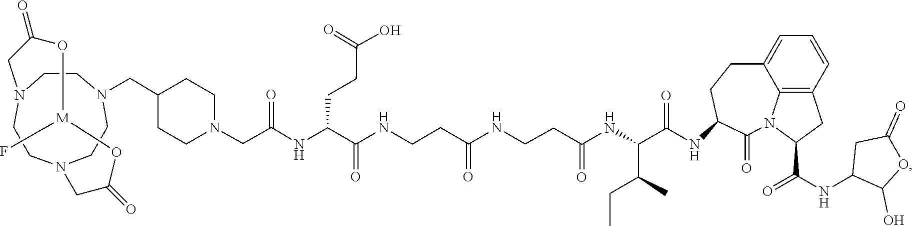
wherein
M is ¹⁷⁷Lu, ⁹⁰Y, ²²⁵Ac, or ²¹³Bi.

13. A pharmaceutical composition comprising a compound of claim 1 and a pharmaceutically acceptable carrier.

14. A method of treating cancer in a subject, the method comprising:
(a) administering to a subject in need thereof an effective amount of a compound of claim 1 or a pharmaceutical composition comprising such.

15. The method of claim 14, further comprising, prior to step (a), administering to the subject an immunotherapeutic agent.

16. The method of claim 14, wherein the immunotherapeutic agent is an immune checkpoint inhibitor, which optionally is a PD1 inhibitor, or a genetically engineered T cell expressing a chimeric antigen receptor (CAR).

17. The method of claim 14, further comprising, prior to step (a), administering to the subject an imaging agent to image granzyme B.

18. The method of claim 14, wherein the subject is further treated with one or more additional therapeutic agents.

19. The method of claim 18, wherein the one or more additional therapeutic agents is selected from the group consisting of anti-inflammatory agents, steroids, immunotherapy agents, and chemotherapeutic agents.

20. A kit for cancer therapy comprising a first container with the compound of claim 1 or a pharmaceutical composition comprising such, and wherein the first container includes a vial, ampule, bottle, syringe, or dispenser package.

21. The kit of claim 20, comprising a pharmaceutical excipient within the second container

22. (canceled)

23. The kit of claim 21, wherein the compound or pharmaceutical composition and the pharmaceutical excipient are combined to form one unit dosage form.

24. The kit of claim 20, comprising an imaging agent suitable for imaging granzyme B.

25. The kit of claim 20, comprising an additional therapeutic agent included in the first container.

26. The kit of claim 25, wherein the additional therapeutic agent includes an immunotherapeutic agent.

27. (canceled)

28. The kit of claim 26, wherein the immunotherapeutic agent is an immune checkpoint inhibitor, which optionally is a PD1 inhibitor, or a genetically engineered T cell expressing a chimeric antigen receptor (CAR).

29. The kit of claim 20, comprising an imaging agent suitable for imaging granzyme B, an additional therapeutic agent, and instructions for using one or more of the compound, pharmaceutical composition, imaging agent, and additional therapeutic agent.

30. The kit of claim 29, comprising instructions for imaging Granzyme B and/or treating and/or reducing the risk of cancer in a subject.

31. (canceled)
